(19) United States
(12) Patent Application Publication
(10) Pub. No.: US 2024/0300922 A1
(43) Pub. Date: Sep. 12, 2024

(54) PROCESS FOR PREPARING EGFR INHIBITORS

(71) Applicant: BLUEPRINT MEDICINES CORPORATION, Cambridge, MA (US)

(72) Inventors: Erika Butler, Halifax (CA); Christopher Lee, Chambridge, MA (US); Lauren MacEachern, Dartmouth (CA); Joshua D. Waetzig, Cambridge, MA (US)

(21) Appl. No.: 18/573,220

(22) PCT Filed: Jun. 22, 2022
(86) PCT No.: PCT/US2022/034487

§ 371 Date: Dec. 21, 2023

Related U.S. Application Data
(60) Provisional application No. 63/214,069, filed on Jun. 23, 2021.

(51) Int. Cl. C07D 401/14

(52) U.S. Cl. CPC C07D 401/14

(57) ABSTRACT

The present disclosure provides methods of producing a compound of structural formula (I): (I) or a pharmaceutically acceptable salt thereof.

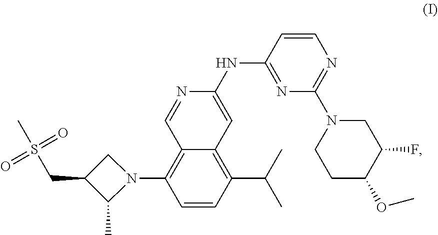 [SMILES: CO[C@@H]1CCN(c2nccc(Nc3cc4c(C(C)C)ccc(N5C[C@H](CS(C)(=O)=O)[C@H]5C)c4cn3)n2)C[C@@H]1F]

### CROSS-REFERENCE TO RELATED APPLICATIONS

This application claims priority to U.S. Provisional Application No. 63/214,069, filed Jun. 23, 2021. The entire contents of the aforementioned application are incorporated herein by reference.

### BACKGROUND

EGFR (Epidermal Growth Factor Receptor) is a member of the erbB receptor family, which includes transmembrane protein tyrosine kinase receptors. By binding to its ligand, such as epidermal growth factor (EGF), EGFR can form a homodimer on the cell membrane or form a heterodimer with other receptors in the family, such as erbB2, erbB3, or erbB4. The formation of these dimers can cause the phosphorylation of key tyrosine residues in EGFR cells, thereby activating a number of downstream signaling pathways in cells. These intracellular signaling pathways play an important role in cell proliferation, survival and anti-apoptosis. Disorders of EGFR signal transduction pathways, including increased expression of ligands and receptors, EGFR gene amplification and alterations such as mutations, deletions and the like, can promote malignant transformation of cells and play an important role in tumor cell proliferation, invasion, metastasis and angiogenesis. For example, alterations such as mutations and deletions in the EGFR gene are found in non-small lung cancer (NSCLC) tumors. The two most frequent EGFR alternations found in NSCLC tumors are short in-frame deletions in exon 19 (del19) and L858R, a single missense mutation in exon 21 (Cancer Discovery 2016 6(6) 601). These two alterations cause ligand-independent EGFR activation and are referred to as primary or activating mutations in EGFR mutant NSCLC (EGFR M+). Clinical experience shows an objective response rate (ORR) of approximately 60-85% in EGFR M+NSCLC patients treated first line (1L) with EGFR tyrosine kinase inhibitors (TKIs) erlotinib, gefitinib, afatinib and osimertinib (Lancet Oncol. 2010 Vol. 11, 121; Lancet Oncol. 2016 Vol. 17, 577; N. Engl. J. Med. 2017 Nov. 18 Doi:10.1056/NEJMoa1713137; Lancet Oncol. 2011 Vol. 12, 735), thus demonstrating that EGFR mutant NSCLC tumors depend on oncogenic EGFR activity for survival and proliferation and establishing del19 and L858R mutated EGFR as oncogenic drivers of disease and thus, validating drug targets and biomarkers for the treatment of NSCLC.

However, after an average of 10-12 months of treatment with first generation (erlotinib and gefitinib) and second generation (afatinib) EGFR TKIs, resistance to these small molecule inhibitors has been observed in almost all NSCLC patients (Lancet Oncol. 2010 February;11(2):121-8; Lancet Oncol. 2016 May; 17(5):577-89; Lancet Oncol. 2011 August; 12(8):735-42). The most prominent resistance mechanism to first and second generation EGFR TKIs is due to the secondary mutation in EGFR of T790M, which occurs in 50% to 70% of patients progressing on 1st and 2nd generation EGFR inhibitors. (Cancer Discov; 2(10); 872-5, 2012; Cancer Res., 65:(16), 2005). This secondary mutation reduces the affinity of the drug with the target, thereby producing drug resistance, and resulting in tumor recurrence or disease progression.

In view of the prevalence of this mutation in drug resistance produced in therapy targeting EGFR of lung cancer, a number of companies have attempted to develop new small molecule EGFR inhibitors for treating these patients with drug-resistant lung cancer by inhibiting the resistant mutant EGFR-T790M. For example, osimertinib (Tagrisso®), a third generation EGFR TKI, has been developed to treat NSCLC patients if the cancer cells are positive for the primary EGFR mutations del19 or L858R with or without the T790M mutation in the gene coding for EGFR.

Although the third generation EGFR TKI, osimertinib, has shown efficacy on NSCLC patients, unfortunately, resistance mediated by an exon 20 C797 mutation in EGFR usually develops within approximately 10 months (European Journal of Medicinal Chemistry 2017 Vol. 142: 32-47) and accounts for the majority of osimertinib resistance cases (Cancer Letters 2016 Vol. 385: 51-54). The EGFR del19/L858R T790M C797S cis mutant kinase variant typically emerges in second line (2L) patients following treatment with osimertinib and is often referred to as “triple mutant” EGFR and it can no longer be inhibited by first, second, or third generation EGFR inhibitors.

The compound of formula (I) is a highly selective inhibitor of EGFR TKI that can inhibit the triple mutant variant. In addition, the compound represented by the formula can inhibit with high selectivity EGFR mutants with the triple mutant, del19/L858R T790M C797S, while at the same time having no or low activity to wild-type EGFR.

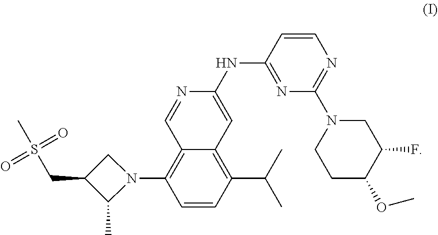 [SMILES: CO[C@@H]1CCN(c2nccc(Nc3cc4c(C(C)C)ccc(N5C[C@H](CS(C)(=O)=O)[C@H]5C)c4cn3)n2)C[C@@H]1F]

### SUMMARY

Provided herein are new methods of making and purifying the compound of formula (I). Further, novel intermediates are disclosed herein.

### DETAILED DESCRIPTION

Disclosed herein are methods of preparing the compound of formula (I). The method comprises reacting a first starting material of formula (Ic):

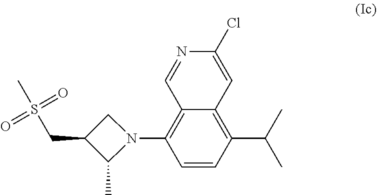 [SMILES: CC(C)c1ccc(N2C[C@H](CS(C)(=O)=O)[C@H]2C)c2cnc(Cl)cc12]

or a salt thereof with a second starting material of formula (Id):

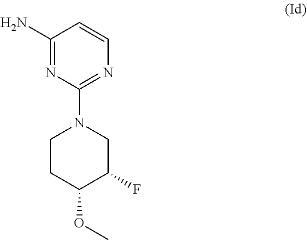 [SMILES: CO[C@@H]1CCN(c2nccc(N)n2)C[C@@H]1F]

or a salt thereof. Also disclosed herein are: i) methods of preparing the compound of formula (Ic) from readily available starting materials; and ii) intermediates obtained from the preparation of the compound of formula (Ic).

The reaction of the starting material of formula (Ic) with the starting material of formula (Id) is in one aspect carried out in the presence of a palladium catalyst and a phosphine ligand. The palladium catalyst and the phosphine ligand are separate compounds. Alternatively, a complex comprises both the palladium catalyst and the phosphine ligand.

A non-limiting list of palladium catalysts includes Pd(dppe)₂ (Bis[1,2-bis(diphenylphosphino)ethane]palladium(0)), CX-11 (1,3-Bis(2,6-diisopropylphenyl)imidazol-2-ylidene(1,4-naphthoquinone)palladium(0) dimer), CX-12 (1,3-Bis(2,4,6-trimethylphenyl)-imidazol-2-ylidene (1,4-naphthoquinone)palladium(0) dimer), Pd(t-Bu₃P)₂ (Bis(tri-tert-butylphosphine)palladium(0)), Pd(PCy₃)₂ (Bis(tricyclohexylphosphine)palladium(0)), Pd(PPh₃)₄ (Tetrakis(triphenylphosphine)palladium(0)), Pd₂(dba)₃ (Tris(dibenzylideneacetone)-dipalladium(0)), Pd(OAc)₂ (Palladium (II) acetate), PdCl₂(PPh₃)₂(Dichlorobis-(triphenylphosphine)palladium(II)), PdCl₂(Amphos)₂ (Bis(di-tert-butyl(4-dimethylaminophenyl)-phosphine)dichloropalladium(II)), Pd(MeCN)₂Cl₂ (Bis(acetonitrile)-dichloropalladium(II)), PdCl₂(P(o-Tol)₃)₂(Dichlorobis(tri-o-tolylphosphine)palladium(II)), Pd(dppf)Cl₂ (1,1′-Bis(diphenylphosphino)ferrocene]-dichloropalladium(II)), Pd(MeCN)₄(BF₄)₂(Tetrakis(acetonitrile)palladium(II) tetrafluoroborate), Pd-PEPPSI-IPent (Dichloro[1,3-bis(2,6-Di-3-pentylphenyl)imidazol-2-ylidene](3-chloropyridyl)palladium(II)), Pd-PEPPSI-IPr ([1,3-Bis(2,6-Diisopropylphenyl)imidazol-2-ylidene](3-chloropyridyl)palladium(II) dichloride), Pd-PEPPSI-SIPr ((1,3-Bis(2,6-Diisopropylphenyl)imidazolidene) (3-chloropyridyl) palladium(II) dichloride), and bis(dibenzylideneacetone)palladium(0) (Pd(dba)₂).

A non-limiting list of phosphine ligands and complexes comprising both the palladium catalyst and the phosphine ligand includes triphenyl phosphine(PPh₃); Bis(tri-o-tolylphosphine) (P(o-Tol)₃)₂; Tri-tert-butoxy phosphine (Pt-Bu₃); Tri-tert-butylphosphonium tetrafluoroborate (Pt-Bu₃HBF₄); Bis(tricyclohexylphosphine (PCy₃); Bis (1-adamanyl)butylphosphane (n-BuP(AD)₂); 2,2′-Bis(diphenylphosphino)-1,1′-binaphthyl (BINAP), (9,9-Dimethyl-9H-xanthene-4,5-diyl)bis(diphenylphosphane)(Xantphos), Bis[(2-diphenylphosphino)phenyl] ether (DPEPhos); 1,1′-Bis(diphenylphosphino)ferrocene (dppf); 1,1′-Bis(di-tert-butylphosphino)ferrocene (dcypf), 1,3-Bis(diphenylphosphino)propane (DPPP), (2-Biphenylyl)di-tert-butylphosphine (JohnPhos), Chloro(2-dicyclohexylphosphino-1,1′-biphenyl)[2-(2′-amino-1,1′-biphenyl)] (CyJohnPhos), 2-Dicyclohexylphosphino-2′-(N,N-dimethylamino)biphenyl (DavePhos), (2-Dicyclohexylphosphino-2′,6′-diisopropoxy-1,1′-biphenyl)[2-(2′-amino-1,1′-biphenyl)] (RuPhos), 2-Dicyclohexylphosphino-2′,6′-dimethoxybiphenyl (SPhos), [(2-Di-cyclohexylphosphino-3,6-dimethoxy-2′,4′,6′-triisopropyl-1,1′-biphenyl)-2-(2′-amino-1,1′-biphenyl)] (BrettPhos), 1,1′-Bis(di-tert-butylphosphino)ferrocene (dtbpf), 2-Di-tert-butylphosphino-2′,4′,6′-triisopropylbiphenyl (t-BuXPhos), [(2-Di-tert-butylphosphino-3,6-dimethoxy-2′,4′,6′-triisopropyl-1,1′-biphenyl)-2-(2′-amino-1,1′-biphenyl)] (t-BuBrettPhos), 2-Di-tert-butylphosphino-3,4,5,6-tetramethyl-2′,4′,6′-triisopropyl-1,1′-biphenyl (Me₄-tBuXPhos), 5-(Di-tert-butylphosphino)-1′,3′,5′-triphenyl-1,1′H-1,4′bipyrazole (BippyPhos), Di(1-adamantyl)-2-morpholinophenylphosphine (MorDalPhos), palladium/1,3-bis-(2,6-diisopropylphenyl)imidazolinium chloride (IPr-HCL), [2-(Di-1-adamantylphosphino)-2′,4′,6′-triisopropyl-3,6-dimethoxybiphenyl][2-(2′-amino-1,1′-biphenyl)]palladium(II) methanesulfonate (AdBrettPhos), (2-Dicyclohexylphosphino-2′,6′-diisopropoxy-1,1′-biphenyl)[2-(2′-amino-1,1′-biphenyl)]palladium(II) methanesulfonate (RuPhos), [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate (BrettPhos), [(2-{Bis[3,5-bis(trifluoromethyl)phenyl]phosphine}-3,6-dimethoxy-2′,4′,6′-triisopropyl-1,1′-biphenyl)-2-(2′-amino-1,1′-biphenyl)]palladium(II) methanesulfonate (JackiePhos), [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate (t-BuBrettPhos), Mesyl(2-(di-tert-butylphosphino)-1,1′-binaphthyl)[2-(2′-amino-1,1′-biphenyl)]palladium (TrixiePhos), (2-Biphenyl)di-tert-butylphosphine, 2′-(Di-tert-butylphosphino)-N,N-dimethylbiphenyl-2-amine (t-BuDavePhos), 2-Di-tert-butylphosphino-2′-methylbiphenyl (t-BuMePhos), Chloro(2-dicyclohexylphosphino-1,1′-biphenyl)[2-(2′-amino-1,1′-biphenyl)]palladium(II) (CyJohnPhos), 2-Dicyclohexylphosphino-2′-methylbiphenyl (MePhos), 2-Dicyclohexylphosphino-2′-(N,N-dimethylamino)biphenyl (PhDavePhos), 2-Dicyclohexylphosphino-2′-methoxy-4′,6′-di-tert-butylbiphenyl (VPhos), 2-[(tert-Butyl)phenylphosphino]-2′,6′-bis(N,N-dimethylamino)biphenyl. (PhCPhos), [(2-Dicyclohexylphosphino-2′,6′-bis(N,N-dimethylamino)-1,1′-biphenyl)-2-(2′-amino-1,1′-biphenyl)] palladium(II) methanesulfonate (CPhos), Methanesulfonato[2-diethylphosphino-2′,6′-bis(dimethylamino)-1,1-biphenyl](2′-amino-1,1′-biphenyl-2-yl)palladium(II) (EtCPhos), 2-Di(tert-butyl)phosphino-2′,4′,6′-triisopropyl-3-methoxy-6-methylbiphenyl (RockPhos), Di-1-adamantyl(4″-butyl-2″,3″,5″,6″-tetrafluoro-2′,4′,6′-triisopropyl-2-methoxy-meta-terphenyl)phosphine (AlPhos), 2-(t-Butylphenylphosphino)-2′,6′-dimethylamino-1,1′-biphenyl, ((t-Bu)PhCPhos), and dicyclohexyl[2′,4′,6′-tris(propan-2-yl)[1,1′-biphenyl]-2-yl]phosphane (XPhos). The foregoing list includes examples where the palladium catalyst and phosphine ligand are part of a complex.

In one aspect, the palladium catalyst and phosphine ligand used in the preparation of the compound of formula (I) is other than the complex methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(II) (BrettPhos-Pd-G4).

In another aspect, the palladium catalyst used in the preparation of the compound of formula (I) is bis(dibenzylideneacetone)palladium(0) (Pd(dba)₂). In another aspect, the phosphine ligand is dicyclohexyl[2′,4′,6′-tris(propan-2-yl)[1,1′-biphenyl]-2-yl]phosphane (XPhos). In yet another aspect, the palladium catalyst used in the preparation of the compound of formula (I) is bis(dibenzylideneacetone)palladium(0) (Pd(dba)₂), and the phosphine ligand is dicyclohexyl[2′,4′,6′-tris(propan-2-yl)[1,1′-biphenyl]-2-yl]phosphane (XPhos).

In another aspect, the reaction mixture further comprises a base. Suitable bases include potassium carbonate (K₂CO₃), cesium carbonate (Cs₂CO₃), potassium hydroxide (KOH), and sodium tert-butoxide (NaOtBu). In another aspect, the base is cesium carbonate (Cs₂CO₃) or sodium tert-butoxide (NaOtBu).

In another aspect, the reaction is carried out in a solvent such as toluene, 1,4-dioxane, tetrahydrofuran (THF), methyl tetrahydrofuran, anisole, water (H₂O), or mixtures thereof. In some examples, the reaction is carried out in 1,4-dioxane, tetrahydrofuran (THF), water (H₂O), or mixtures thereof. In some examples, the reaction is carried out in 1,4-dioxane, toluene, or mixtures thereof.

In one aspect, the compound of formula (I) can be purified by recrystallizing in, for example, a solvent system such as dimethyl sulfoxide (DMSO) and ethanol. For example, the compound of formula (I) can be dissolved in dimethyl sulfoxide (DMSO), optionally with heating, and then ethanol (or water) may be added, optionally with cooling. In another aspect, seed crystal(s) of the compound of formula (I) can be added to facilitate the crystallization. In one aspect, the compound of formula (I) is obtained from the methods described above or in the Exemplification and is isolated from the reaction as, for example, a wet cake.

Specific conditions for preparing the compound of formula (I) from the compounds of formulas (Ic) and (Id) are provided in Example 4.

Also disclosed herein is the preparation of the compound of formula (Ic).

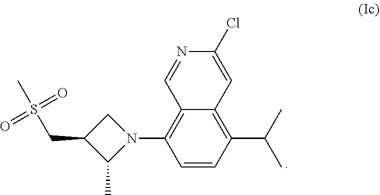 [SMILES: CC(C)c1ccc(N2C[C@H](CS(C)(=O)=O)[C@H]2C)c2cnc(Cl)cc12]

As noted above, the compound of formula (Ic) is a starting material used in the preparation of the compound of formula (I). The method of preparing the compound of formula (Ic) comprises reacting a first starting material of formula (Ia):

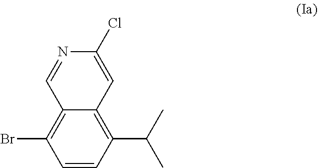

or a salt thereof, with a second starting material of formula (Ib):

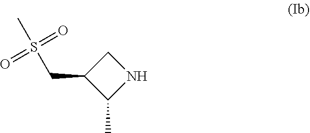

or a salt thereof, in the presence of a base, a palladium catalyst, and a phosphine ligand to form the compound of formula (Ic). Suitable palladium catalyst and phosphine ligands are as described above for the preparation of the compound of formula (I). Suitable bases are as described above for the preparation of the compound of formula (I).

In one aspect, the base in the reaction between starting material (Ia) and (Ib) is cesium carbonate (Cs₂CO₃), the palladium catalyst is bis(dibenzylideneacetone)palladium(0) (Pd(dba)₂), and the phosphine ligand is (9,9-dimethyl-9H-xanthene-4,5-diyl)bis(diphenylphosphane) (Xantphos). In some examples, the reaction is carried out in a polar solvent, such as dioxane. The reaction may also be conducted with heating, such as at 90° C. to 110° C., or at 92° C. to 108° C., or at 95° C. to 105° C.

In one aspect, the base in the reaction between starting material (Ia) and (Tb) is potassium hydroxide (KOH), the palladium catalyst is bis(dibenzylideneacetone)palladium(0) (Pd(dba)₂), and the phosphine ligand is (9,9-dimethyl-9H-xanthene-4,5-diyl)bis(diphenylphosphane) (Xantphos). In some examples, the reaction is carried out in a nonpolar solvent, such as toluene. The reaction may also be conducted with heating, such as at 70° C. to 110° C., or at 80° C. to 100° C., or at 85° C. to 95° C.

In one aspect, compound (Ic) prepared by the methods described above is reacted with the compound of formula (Id) without isolating the compound of formula (Ic).

Specific conditions for preparing compound (Ic) are provided in Example 4.

Also disclosed herein is a method of preparing the compound of formula (Ta). As described above, the compound of formula (Ia) is a starting material used in the preparation of the compound of formula (Ic). The preparation of the compound of formula (Ia) is a five step procedure, each of which is described below. Each reaction step is considered to be a separate embodiment. Combinations of these reaction steps, including the combined five step procedure of producing the compound of formula (Ia) are considered to be separate embodiments as well.

The first step in the preparation of the compound of formula (Ia) is a method of preparing a compound of formula (III):

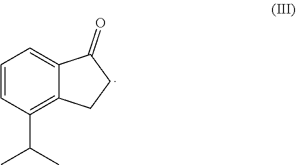

The method comprises hydrogenating a starting material of formula (II):

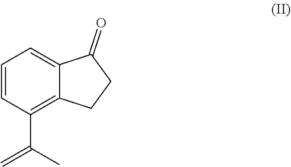

in the presence of a platinum hydrogenolysis catalyst or a palladium hydrogenolysis catalyst to form the compound of formula (III). Suitable hydrogenolysis catalysts include 20% palladium hydroxide on carbon (Perlman's catalyst), palladium chloride, palladium, wet palladium/carbon, and platinum oxide (PtO₂). In one aspect, the platinum hydrogenolysis catalyst is PtO₂ and the palladium hydrogenolysis catalyst is wet palladium/carbon. In another aspect, the reaction is carried out in ethyl acetate (EtOAc) at 20° C. to 30° C., or at 22° C. to 28° C. The compound of formula (II) can be prepared from 4-bromo-indanone (see Example 1.1), which is a known compound (CAS 15115-60-3), which is also commercially available from Sigma Aldrich (Catalog No. 644366).

The second step in the preparation of the compound of formula (Ia) is a method of preparing a compound of formula (IV):

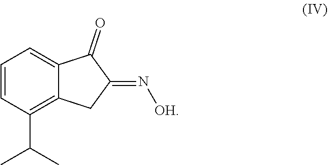

The method comprises reacting a starting material of formula (III):

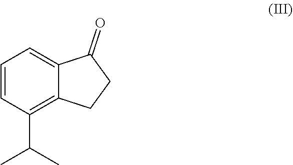

with tert-butyl nitrite (t-BuONO) and hydrogen chloride to form the compound of formula (IV). In one aspect, the reaction is carried out in tetrahydrofuran (THF) at 0° to 10° C., and the hydrogen chloride is methanolic hydrogen chloride. In another aspect, the starting material of Structural Formula (III) is prepared as described in the first step.

The third step in the preparation of the compound of formula (Ia) is a method of preparing a compound of formula (V):

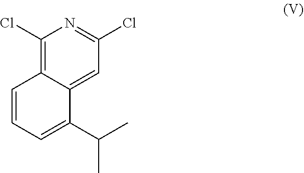

or a salt thereof.

The method comprises reacting a starting material of formula (IV):

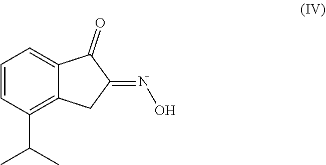

or a salt thereof with phosphoryl chloride (POCl₃), phosphorus pentachloride (PCl₅), and hydrogen chloride to form the compound of formula (V).

In one aspect, the starting material of formula (IV) is combined with POCl₃ and PCl₅ at 0° C. to 25° C. or 5° C. to 20° C., or 10° C. to 15° C., followed by addition of hydrogen chloride and warming to 50° C. to 70° C. or 55° C. to 65° C. In one aspect, the reaction is carried out in dioxane. In another aspect, the starting material of structural formula (IV) is prepared as described in the second step.

The fourth step in the preparation of the compound of formula (Ia) is a method of preparing a compound of formula (VI):

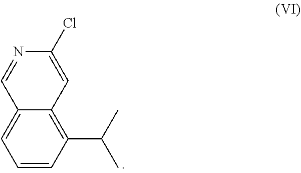

The method comprises reacting a starting material of formula (V):

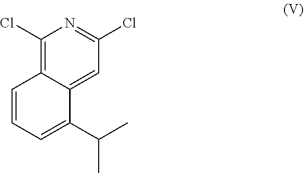

or a salt thereof in the presence of an amine base, a hydride reducing agent, and a palladium catalyst to form the compound of formula (VI).

Amine bases are nitrogen-containing compounds capable of accepting a proton. Examples include methylamine (CH₃NH₂), dimethylamine ((CH₃)₂NH), triemethylamine ((CH₃)₃N), and the C₂-C₆ alkylamine analogues thereof, aniline (PhNH₂) and its derivatives, N,N-diisopropylethylamine, dimethylaminopyridine (DMAP), tetramethylethylenediamine (TMEDA), and pyridine.

A hydride reducing agent is a chemical compound than can reduce the compound of interest by addition of a negatively charged hydrogen ion (H⁻ ion). Examples included sodium hydride (NaH), lithium hydride (LiH), lithium aluminum hydride (LiAlH₄), sodium triethylborohydride, and sodium borohydride (NaBH₄).

Suitable palladium catalyst are as described above for the first embodiment. In one aspect, the palladium catalyst is 1,1′-Bis(diphenylphosphino)ferrocene]-dichloropalladium(II) (Pd(dba)₂), the hydride reducing agent is sodium borohydride and the amine base is tetramethylethylenediamine (TMEDA). In one aspect, the reaction is carried out in tetrahydrofuran and at 20° C. to 30° C. In another aspect, the starting material of formula (V) is prepared as described in the third step.

The fifth step in the preparation of the compound of formula (Ia) comprises reacting a starting material of formula (VI):

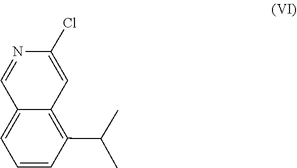

with a brominating agent in an acid to form the compound of formula (Ia).

Suitable acids include, but are not limited to sulfuric acid, methane sulfonic acid, triflic acid, and the like.

A brominating agent is a compound that is capable of adding an electrophilic bromine atom (Br⁺) to a compound of interest. Suitable brominating agents are cyanogen bromide (CNBr), bromine (Br₂) and N-bromosuccinimide (NBS). In one aspect, the brominating agent is N-bromosuccinimide (NBS) and the acid is sulfuric acid (H₂SO₄). In another aspect, the starting material of formula (VI) is prepared as described in the fourth step.

The five step procedure for preparing the compound of formula (Ia) is shown schematically in Example 1. Specific conditions for each of these reaction steps is provided in Example 1.

Also disclosed herein is a method of preparing the compound of formula (Ib):

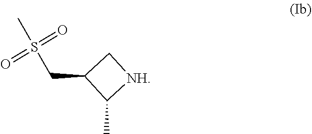

As described above, the compound of formula (Ib) is a starting material used in the preparation of the compound of formula (Ic). The preparation of the compound of formula (Ib) is a five step procedure, each of which is described below. Each reaction step is considered to be a separate embodiment. Combinations of these reaction steps, including the combined five step procedure of producing the compound of formula (Ib) are considered to be separate embodiments as well.

The first step in the preparation of the compound of formula (Ib) is method of preparing a compound of formula (VII):

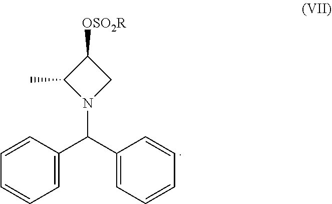

The definition of R is provided below. The method comprises reacting a starting material of formula (VIIa):

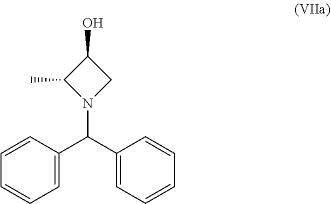 [SMILES: C[C@@H]1[C@@H](O)CN1C(c1ccccc1)c1ccccc1]

with a sulfonyl chloride, e.g., ethanesulfonyl chloride (also referred to as esyl chloride or EsCl), and an amine base, such as triethylamine (TEA), to form the compound of formula (VII).

A sulfonyl chloride has the general formula RSO₂Cl, wherein R is a C₁-C₄ straight or branched alkyl group, or a phenyl group optionally substituted with halogen, a C₁-C₄ alkyl group, and/or a nitro group, or the like. Examples include benzene sulfonyl chloride, tosyl chloride (para-toluenesulfonyl chloride), brosyl chloride (para-bromophenyl sulfonyl chloride), nosyl chloride (nitrophenyl sulfonyl chloride), mesyl chloride (methyl sulfonyl chloride), and esyl choride (ethyl sulfonyl chloride). A sulfonyl group is represented by RSO₂—. In one aspect, the reaction is carried out in dichloromethane at 5° C. to 20° C. or at 10° C. to 15° C.

Suitable amine bases are as described above for the preparation of the compound of formula (VI).

The starting material of formula (VIIa) can obtained according to procedures described in Frigola et al., J. Med. Chem., 38:1203 (1995), the entire teachings of which are incorporated herein by reference.

The second step in the preparation of the compound of formula (Ib) is a method of preparing a compound of formula (VIII):

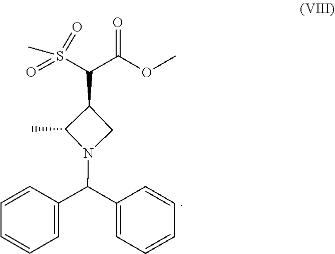 [SMILES: COC(=O)C([C@H]1CN(C(c2ccccc2)c2ccccc2)[C@@H]1C)S(C)(=O)=O]

The method comprises reacting a first starting material of formula (VII):

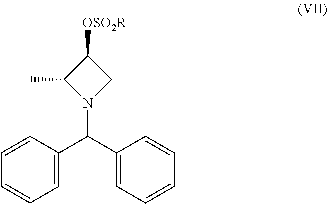

(e.g.,

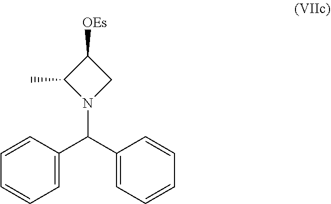

or a salt thereof with a second starting material of formula (VIIIb):

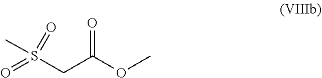

and a base such as potassium carbonate (K₂CO₃) to form the compound of formula (VIII). R is as described above for the Compound of Formula (VII). In another aspect, the starting material of formula (VII) is prepared as described in the first step.

The third step in the preparation of the compound of formula (Ib) is a method of preparing the second starting material of formula (VIIIb). The method comprises reacting methyl 2-bromoacetate with

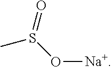

The fourth step in the preparation of the compound of formula (Ib) is a method of preparing a compound of formula (IX):

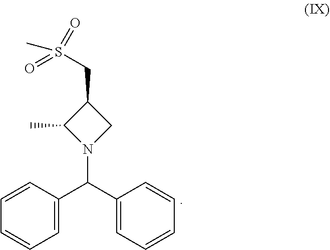 [SMILES: C[C@@H]1[C@@H](CS(C)(=O)=O)CN1C(c1ccccc1)c1ccccc1]

The method comprises reacting a starting material of formula (VIII):

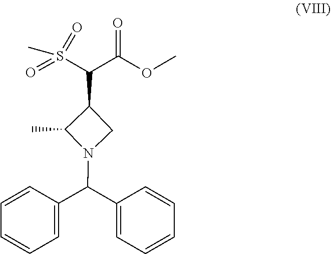 [SMILES: COC(=O)C([C@H]1CN(C(c2ccccc2)c2ccccc2)[C@@H]1C)S(C)(=O)=O]

or a salt thereof with lithium chloride (LiCl) in the presence of water to form the compound of formula (IX). For example, 0.4-0.6 mol equivalents of water may be used. In one aspect, the reaction is carried out in dimethylacetamide (DMAc) at 160° C. to 170° C. In another aspect, the starting material of formula (VIII) is prepared as described in the third step.

The fifth step in the preparation of the compound of formula (Ib) or a salt thereof comprises hydrogenating a starting material of formula (IX):

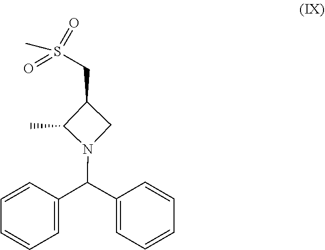 [SMILES: C[C@@H]1[C@@H](CS(C)(=O)=O)CN1C(c1ccccc1)c1ccccc1]

or a salt thereof in the presence of a palladium hydrogenolysis catalyst to form the compound of formula (Ib). In one aspect, the palladium hydrogenolysis catalyst is palladium hydroxide on carbon, 20 wt. % dry basis (20% Pd(OH)₂/C) and the reaction is carried out in methanol (MeOH) at 30° C. to 50° C. or at 35° C. to 45° C. In another aspect, the starting material of formula (IX) is prepared as described in the fourth step.

The five-step procedure for preparing the compound of formula (Ib) is shown schematically in Example 2. Specific conditions for each of these reaction steps is provided in Example 2.

Another embodiment of the disclosure is a compound selected from:

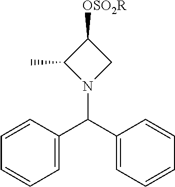

wherein R as defined herein, e.g.,

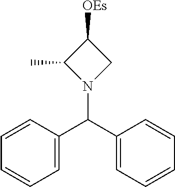

(Es is ethyl sulfonyl),

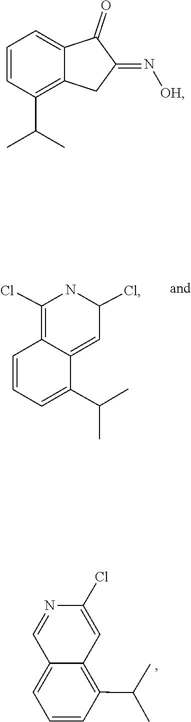

or a salt of any of the foregoing.

These compounds possess a basic group and accordingly can react with inorganic and organic acids, to form a salt. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

### EXEMPLIFICATION

### Preparation of Exemplary Compounds

### Definitions

ACN acetontirile
C Celsius
Cs₂CO₃ cesium carbonate
DCM dichloromethane
DMAc dimethylacetamide
DMSO dimethylsulfoxide
EsCl ethanesulfonyl chloride
EtOAc ethyl acetate
EtOH ethanol
g gram
h hour
H₂ hydrogen
H₂O water
H₂SO₄ sulfuric acid
HCl hydrogen chloride
HPLC high performance liquid chromatography
IC₅₀ inhibitory concentration 50%
LC-MS liquid chromatography-mass spectroscopy
LiCl lithium chloride
K₂CO₃ potassium carbonate
kg kilogram
mbar milli bar
MeOH methanol
min minutes
MTBE methyl tert-butyl ether
N₂ nitrogen
NaBH₄ sodium borohydride
NaHSO₃ sodium bisulfite
Na₂SO₄ sodium sulfate
NLT no less than
NMT no more than
PtO₂ platium oxide
R1 first reactor
R2 second reactor
RT retention time
rt room temperature
SiO₂ silicon dioxide
t-BuONO tert-butyl nitrite
TEA triethylamine
TLC thin layer chromotography
TMEDA tetramethylethylenediamine
THF tetrahydrofuran
*3 number of repeats (e.g., 3 times)

LC-MS: The liquid chromatography-mass spectrometry (LC-MS) data (sample analyzed for purity and identity) were obtained with an Agilent model-1260 LC system using an Agilent model 6120 mass spectrometer utilizing ES-API ionization fitted with an Agilent Poroshel 120 (EC-C18, 2.7 um particle size, 3.0×50 mm dimensions) reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 4 minutes was utilized. The flow rate was constant at 1 mL/min.

Alternatively, the liquid chromatography-mass spectrometry (LC-MS) data (sample analyzed for purity and identity) were obtained with a Shimadzu LCMS system using an Shimadzu LCMS mass spectrometer utilizing ESI ionization fitted with an Agilent (Poroshel HPH-C18 2.7 um particle size, 3.0×50 mm dimensions) reverse-phase column at 22.4 degrees Celsius. The mobile phase consisted of a mixture of solvent 5 mM NH₄HCO₃ (or 0.05% TFA) in water and acetonitrile. A constant gradient from 90% aqueous/10% organic to 5% aqueous/95% organic mobile phase over the course of 2 minutes was utilized. The flow rate was constant at 1.5 mL/min.

Silica gel chromatography: Silica gel chromatography was performed on a Teledyne Isco CombiFlash® Rf unit, a Biotage® Isolera Four unit, or a Biotage® Isolera Prime unit.

Proton NMR: ¹H NMR spectra were obtained with a Varian 400 MHz Unity Inova 400 MHz NMR instrument (acquisition time=3.5 seconds with a 1 second delay; 16 to 64 scans) or a Avance 400 MHz Unity Inova 400 MHz NMR instrument (acquisition time=3.99 seconds with a 1 second delay; 4 to 64 scans) or a Avance 300 MHz Unity Inova 300 MHz NMR instrument (acquisition time=5.45 seconds with a 1 second delay; 4 to 64 scans). Unless otherwise indicated, all protons were reported in DMSO-d6 solvent as parts-per million (ppm) with respect to residual DMSO (2.50 ppm).

GC: Gas chromatographs were obtained with an Agilent 7890C gas chromatograph or similar with a DB-1 15 m×0.25 mm×1.0 μm or equivalent column, with an injector temperature of 250° C., a detector temperature of 325° C., and a constant flow of nitrogen carrier gas of 1.6 mL/min.

### SYNTHETIC EXAMPLES

### Example 1: Synthesis of 8-bromo-3-chloro-5-isopropylisoquinoline (Ia)

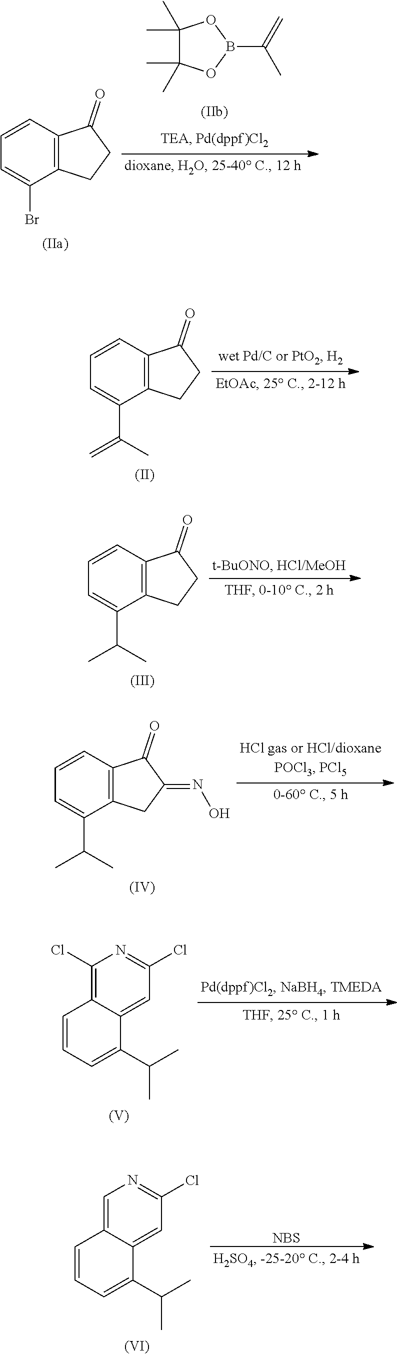

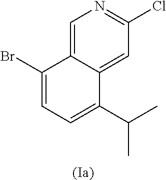

### 1.1 Preparation of 4-(prop-1-en-2-yl)-2,3-dihydro-1H-inden-1-one (II)

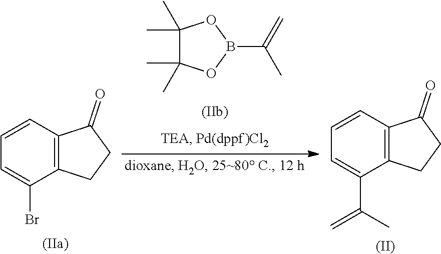

To a solution of compound (IIa) (500 g, 2.37 mol, 1.00 eq) in dioxane (2500 mL) and H₂O (500 mL) was added compound (IIb) (398 g, 2.37 mol, 1.00 eq), Pd(dppf)Cl₂ (17.3 g, 23.6 mmol, 0.01 eq) and TEA (719 g, 7.11 mol, 989 mL, 3.00 eq) at 25° C. The reaction mixture was stirred at 80° C. for 12 h. LCMS showed compound (IIa) was consumed completely and the desired mass (RT=0.885 min) was detected. Three batches were combined. The mixture was filtered through celite and the filter cake was washed with ethyl acetate (500 mL*3). H₂O (4000 mL) was added to the filtrate and extracted with ethyl acetate (1000 mL*3). The organic phase was washed with brine (2000 mL), dried over Na₂SO₄, filtered and concentrated to get the residue. The residue was purified by column chromatography (SiO₂), Petroleum Ether/Ethyl Acetate=50/1 to 10/1, Rf=0.4). Compound (II) (1.05 kg, 6.05 mol, 85.1% yield, 99.0% purity) was obtained as light yellow solid and confirmed via ¹H NMR and LCMS.

LC-MS: product: RT=0.885 min, m/z=173.0 (M+H)⁺.

¹HNMR: (400 MHz, CDCl₃) [ppm] δ 7.68 (dd, J=7.6, 0.8 Hz, 1H), 7.49 (dd, J=8.0, 1.2 Hz, 1H), 7.34-7.38 (m, 1H), 5.30-5.31 (m, 1H), 5.10 (d, J=1.2, 0.8 Hz, 1H), 3.15-3.18 (m, 2H), 2.67-2.71 (m, 2H), 2.14-2.15 (m, 3H).

### 1.2 Preparation of 4-isopropyl-2,3-dihydro-1H-inden-1-one (III)

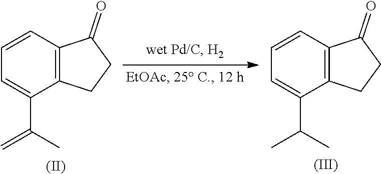

To a solution of compound (II) (1.05 kg, 6.04 mol, 1.00 eq) in EtOAc (10.5 L) was added wet Pd/C (210 g, 10% Pd content) at 25° C. under N₂. The suspension was degassed under vacuum and purged with H₂ several times. The mixture was stirred under H₂ (20 psi) at 25° C. for 12 h. LCMS showed compound (II) was consumed completely and the desired mass (RT=0.802 min) was detected. The mixture was filtered through celite and washed with ethyl acetate (2000 mL*3). The filtrate was concentrated to get the residue. The residue was used to next step without further purification. Compound (III) (1.08 kg, crude) was obtained as white solid and confirmed via LCMS.

LC-MS: product: RT=0.858 min, m/z=175.1 (M+H)⁺.

### 1.3 Preparation of (E)-2-(hydroxyimino)-4-isopropyl-2,3-dihydro-1H-inden-1-one (IV)

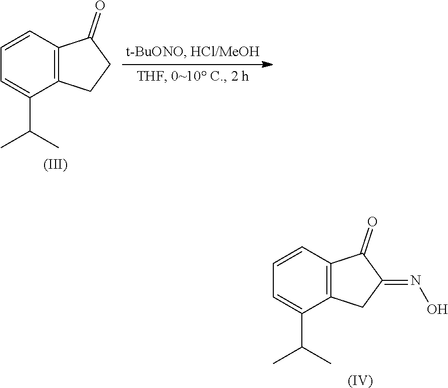

To a solution of compound (III) (295 g, 1.69 mol, 1.00 eq) in THF (750 mL) was added t-BuONO (262 g, 2.54 mol, 302 mL, 1.50 eq) at 0-10° C. under N₂. Then HCl/MeOH (4 M, 110 mL, 0.26 eq) was drop-wisely added to the mixture at 0-10° C. After the addition, the reaction mixture was stirred at 0° C. for 2 h. LCMS showed that compound (III) was consumed and desired mass (RT=0.774 min) was detected. The reaction mixture was concentrated to get the residue. The residue was slurried with Petroleum Ether/Ethyl Acetate=7/1 (800 mL) and filtered, the filter cake was collected to get the light yellow solid. Compound (IV) (205 g, 1.00 mol, 59.2% yield, 99.4% purity) was obtained as light yellow solid, which was confirmed by LCMS and ¹H NMR.

LC-MS: product: RT=0.773 min, m/z=204.1 (M+H)⁺.

¹H NMR: (400 MHz, DMSO) δ [ppm] 12.65 (s, 1H), 7.65 (d, J=7.6 Hz, 1H), 7.58 (d, J=7.2 Hz, 1H), 7.46 (t, J=7.6 Hz, 1H), 3.77 (s, 2H), 3.06-3.36 (m, 1H), 1.24 (d, J=6.8 Hz, 6H).

### 1.4 Preparation of 1,3-dichloro-5-isopropylisoquinoline (V)

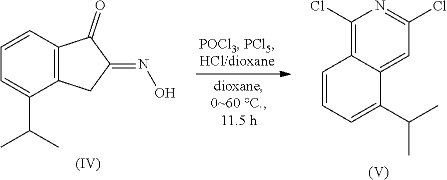

To a solution of compound (IV) (133 g, 650 mmol, 1.00 eq) in dioxane (650 mL) was added POCl₃ (151 g, 984 mmol, 91.5 mL, 1.51 eq) at 25° C. Then PCl₅ (203 g, 976 mmol, 1.50 eq) was added to the mixture at 0-20° C. in portions. The mixture was stirred at 0-20° C. for 0.5 h. Then HCl/dioxane (4 M, 16.3 mL, 0.10 eq) was added to the mixture at 0-20° C., and then the mixture was stirred at 60° C. for 11 h. LCMS showed compound (IV) was consumed completely and the desired mass (RT=1.074 min) was detected. The mixture was quenched with H₂O (1500 mL) and extracted with dichloromethane (300 mL*3). The organic phase was washed with brine (300 mL), dried over Na₂SO₄, filtered and concentrated to get the residue. The residue was combined and was purified by column chromatography (SiO₂, Petroleum Ether/Ethyl Acetate=1/0 to 100/1, R_{f}=0.35). The residue was detected by TLC (Petroleum Ether/Ethyl Acetate=1/0, R_{f}=0.35). Compound (V) (138 g, 575 mmol, 64.5% yield) was obtained as yellow oil and confirmed by LCMS.

LC-MS: product: RT=1.074 min, m/z=239.9 (M+H)⁺

### 1.5 Preparation of 3-chloro-5-isopropylisoquinoline (VI)

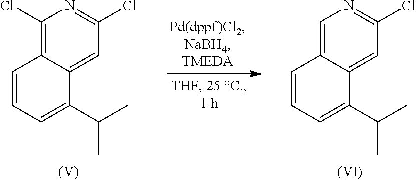

To a solution of compound (V) (170 g, 581 mmol, 1.00 eq) in THF (850 mL) was added Pd(dppf)Cl₂ (4.25 g, 5.81 mmol, 0.01 eq) at 25° C. under nitrogen. Then TMEDA (101 g, 872 mmol, 132 mL, 1.50 eq) and NaBH₄ (81.6 g, 2.16 mol, 3.71 eq) was added to the mixture. The reaction mixture was stirred at 25° C. for 1 h. TLC (Petroleum Ether/Ethyl Acetate=10/1) showed compound (V) (R_{f}=0.8) was consumed completely and the major spot (R_{f}=0.6) was detected. The mixture was poured into cooled 1N HCl aqueous (1000 mL) and extracted with ethyl acetate (500 mL*3). The organic phase was filtered through celite and the filtrate was washed with brine (200 mL), dried over anhydrous Na₂SO₄, filtered and concentrated to get the residue. The residue was purified by column chromatography (SiO₂, Petroleum Ether/Ethyl Acetate=1/0 to 100/1, R_{f}=0.6). Compound (VI) (137 g, crude) was obtained as yellow oil and confirmed via ¹H NMR and LCMS.

LC-MS: product: RT=0.901 min, m/z=206.1 (M+H)⁺.

¹H NMR: (400 MHz, CDCl₃) δ [ppm] 9.06 (s, 1H), 7.94 (s, 1H), 7.82 (d, J=8.0 Hz, 1H), 7.64 (d, J=6.8 Hz, 1H), 7.55-7.58 (m, 1H), 3.55-3.65 (m, 1H), 1.40 (d, J=6.8 Hz, 6H).

### 1.6 Preparation of 3-chloro-5-isopropylisoquinoline (Ia)

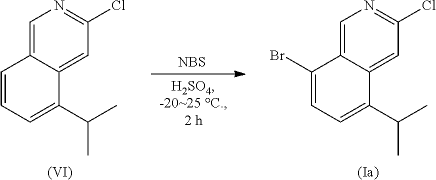

Compound (VI) (93.8 g, 392 mmol, 1.00 eq) was added to the solution of H₂SO₄ (500 mL) at −10-0° C. After the addition, the mixture was cooled down to −10˜−20° C. and NBS (90.7 g, 510 mmol, 1.30 eq) was added to the mixture at −10˜−20° C. Then the reaction mixture was stirred at 25° C. for 2 h. TLC (Petroleum Ether/Ethyl Acetate=20/1) showed compound (VI) (R_{f}=0.6) remained and the major spot (R_{f}=0.9) was formed. The mixture was poured into ice (1500 g) at 0-10° C. and adjusted to pH=9 with ammonium hydroxide (1800 mL), then extracted with ethyl acetate (500 mL*2). The organic phase was washed with brine (1000 mL), dried over Na₂SO₄, filtered and concentrated to get the residue. The residue was purified by column chromatography (SiO₂, Petroleum Ether/Ethyl Acetate=1/0 to 50/1, R_{f}=0.9). Compound (VI) (70.03 g, 230 mmol, 58.7% yield, 93.6% purity) was obtained as off-white solid and confirmed via ¹H NMR), LCMS, and HPLC).

LC-MS: product: RT=1.149 min, m/z=283.9 (M+H)⁺.

HPLC: product: RT=2.863 min, 93.6% purity under 220 nm.

¹HNMR: (400 (MHz, CDCl₃) δ [ppm] 9.43 (s, 1H), 7.91 (s, 1H), 7.78 (d, J=7.6 Hz, 1H), 7.46 (d, J=7.6 Hz, 1H), 3.51-3.62 (m, 1H), 1.38 (d, J=7.2 Hz, 6H).

### Example 2: Synthesis of (2R,3S)-2-methyl-3-((methylsulfonyl)methyl) azetidine hydrochloride (Ib)

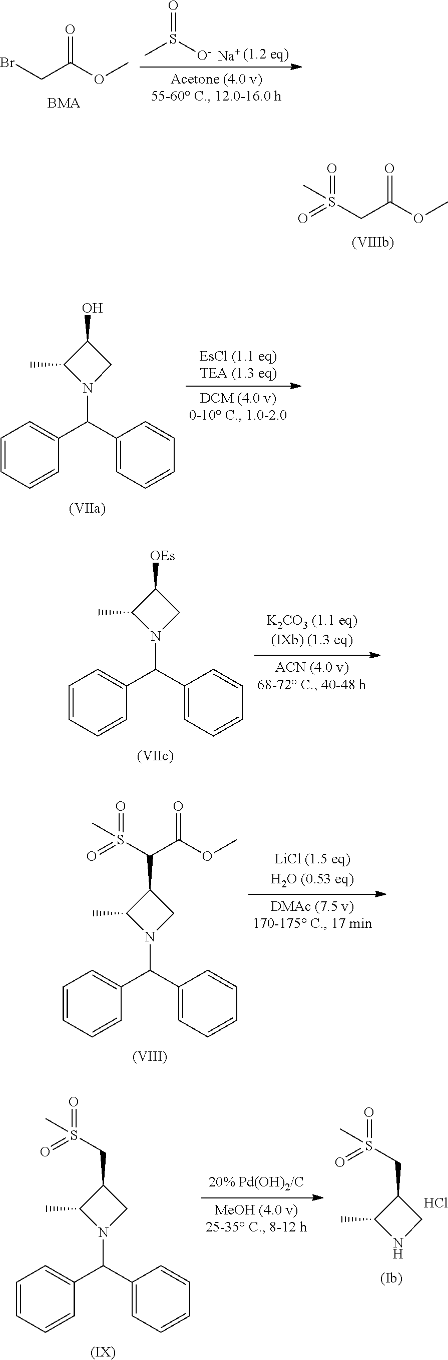

### 2.1 Preparation of methyl 2-(methylsulfonyl)acetate (VIIIb)

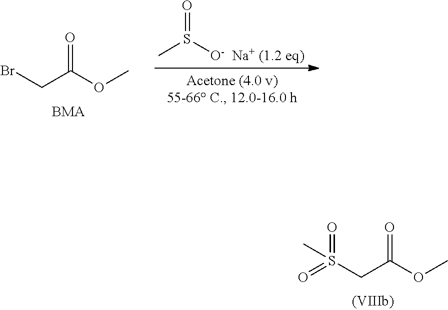

To a 3000 L reactor was charged sodium methyl sulfinate (153.19 kg, 1500 mol, 1.2 eq) and acetone (760.00 kg), 2-bromomethyl acetate (BMA) (190.00 kg, 1250 mol, 1.0 eq) was added in one portion. The reaction mixture was heated to 55-60° C. and stirred at 55-60° C. over 12-16 hours. Upon reaction completion (GC monitoring), the reaction mixture was cooled to 15-20° C. The reaction mixture was filtered, and the filter cake was washed once with acetone (50 L). The combined filtrate was concentrated under vacuum to 300-350 L volume. n-Heptane (200 L) was added, and the mixture continued to concentrate to 300-350 L volume. This operation was repeated twice to remove residual acetone.

n-Heptane (400 L) was charged, and the mixture was stirred at 20-30° C. for 1-2 h. The mixture was filtered, and the filter cake was washed once with n-Heptane (60 L). The wet cake was dried at 35-40° C. for 6-10 h to afford compound (VIIIb) as a white solid (167.60 kg, 88.2% yield), GCAP:100%.

GC Purity: 100% (a/a), RT=3.79 min.

¹H NMR: (400 MHz, CDCl₃) δ [ppm] 4.02 (s, 2H), 3.85 (s, 3H), 3.16 (s, 2H).

### 2.2 Preparation of (((2R,3S)-1-benzhydryl-methylazetidin-3-yl)oxy)esylate (VIIc)

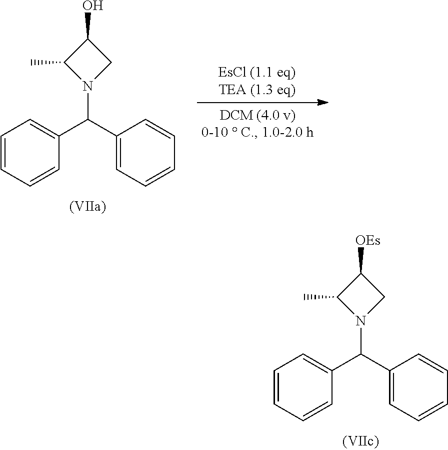

To a 2000 L reactor was charged compound (VIIa) (215.00 kg, 848.7 mol, 1.0 eq) and DCM (1144.00 kg). After stirring for 5 min, TEA (111.64 kg, 1103.3 mol, 1.3 eq) was added. The reaction mixture was cooled to 0-10° C. under the protection of nitrogen. EsCl (120.03 kg, 933.5 mol, 1.1 eq) was added slowly to the reaction over 2-3 hours, while keeping the temperature at <10° C. White solids formed during the addition. The reaction was stirred for another 1-2 hours after addition.

The reaction mixture was quenched with H₂O (645 L) and stirred for 15 min. The organic layer was separated, and the aqueous phase was extracted once with DCM (215 L). The combined organic phase was washed with 10% brine (215 L). The organic portion was concentrated under vacuum at 40-45° C. to 450-500 L volume. n-Heptane (645 L) was added, and the mixture was distilled to 450-500 L volume. This operation was repeated twice to remove residual DCM. n-Heptane (645 L) was charged, and the mixture was stirred at 20-30° C. for 1-2 h. The mixture was filtered, and the filter cake was washed once with n-Heptane (88 L), and the wet cake was dried at 45-55° C. under vacuum for 6-10 h to afford compound (VIIc) as a yellow solid (284.20 kg, 96.9% yield).

HPLC Purity: 99.7% (a/a), RT=6.70 min.

¹H NMR: (400 MHz, CDCl₃) δ [ppm] 7.45-7.33 (m, 4H), 7.30-7.21 (m, 6H), 4.66-4.61 (ddd, 1H), 4.43 (s, 1H), 3.77-3.73 (dd, 1H), 3.43-3.36 (dq, 1H), 3.43-3.37 (q, 2H), 2.91-2.87 (dd, 1H), 1.43-1.40 (t, 3H), 0.84-0.83 (d, 3H).

### 2.3 Preparation of methyl (S)2-(2R,3S)-1-benzyhydryl-2-methylazetidin-3-yl)-2-((methylsulfonyl)acetate) (VIII)

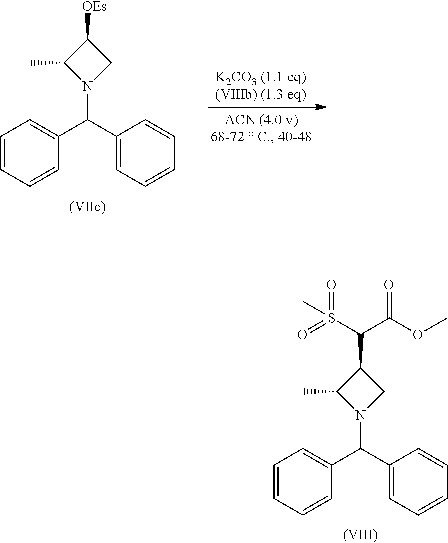

To a 2000 L reactor was added compound (VIIc) (142.2 kg, 411.6 mol, 1.0 eq) and acetonitrile (665.50 kg). After stirring 5 min, methyl 2-(methylsulfonyl)acetate compound (VIIIb) 75.16 kg, 494.0 mol, 1.2 eq) and K₂CO₃ (113.78 kg, 823.3 mol, 2.0 eq) were added separately. The reaction mixture was heated to 68-72° C. and stirred at 68-72° C. over 16 hours. K₂CO₃ (28.45 kg, 205.8 mol, 0.5 eq) was added to the reaction mixture, which was then stirred for 24 hours at 68-72° C. Upon reaction completion (HPLC monitoring), the reaction mixture was cooled to 15-20° C. The reaction mixture was centrifuged, the filtrate was concentrated to 150-200 L volume, and the filtrate was combined with the filtrate in next step.

The centrifugal filter cake was suspended in ethyl acetate (570 L) and stirred for 1-2 hours. The slurry was centrifuged. The filtrate was combined with the filtrate from the previous step. A prepared 10% NaCl aqueous solution (156 L) was added to the combined liquid and stirred for 15 min. The organic layer was separated, the aqueous phase was extracted with ethyl acetate (142 L). The combined organic phase was washed twice with 10% brine (142 L×2). The organic phase was concentrated under reduced pressure at 45-55° C.

MTBE (284 L) was added to the residue and stirred for 1 to 2 hours, then n-heptane (383 L) was added slowly for 2-3 hours at 15-20° C. The resulting slurry was filtered, and the filter cake was washed once with n-heptane (50 L). The wet cake was dried at 45-55° C. under vacuum for 6-10 h to afford compound (VIII) as a yellow solid (125.70 kg, 78.9% yield).

HPLC Purity: 97.7% (a/a), RT=6.30 min.

¹H NMR: (400 MHz, DMSO-d₆) δ [ppm] 7.43-7.27 (m, 4H), 7.25-7.18 (m, 6H), 4.69-4.59 (dd, 1H), 4.50 (s, 1H), 3.74 (s, 3H), 3.45-3.40 (ddd, 1H), 3.35-3.15 (m, 1H), 3.05 (d, 3H), 2.73-2.55 (m, 2H), 0.76-0.58 (dd, 3H).

### 2.4 Preparation of (2R,3S)-1-benzyhydryl-2-methyl-3-((methylsulfonyl)methyl) azetidine (IX)

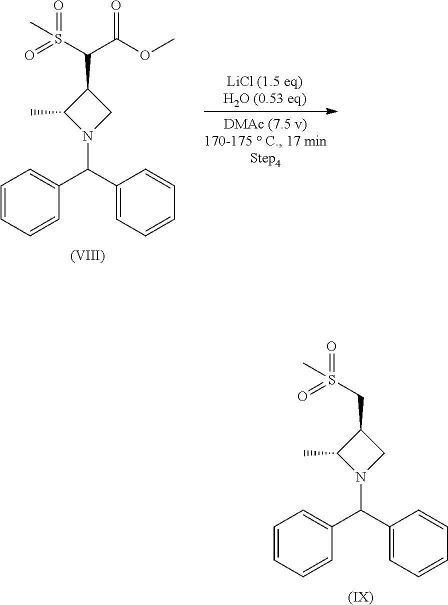

To a reactor was charged compound (VIII) (120.8 kg, 311.7 mol, 1.0 eq) and DMAc (849.00 kg), then LiCl (19.82 kg, 467.6 mol, 1.5 eq) and H₂O (3.00 kg, 166.7 mol, 0.53 eq) was added, the mixture was stirred until dissolved. The above mixture was reacted in a flow reactor at 170-175° C., reaction time is 17 minutes. The reaction mixture was monitored by HPLC every 1-2 hours. Water (725 L) and ethyl acetate (966 L) were added to the reaction mixture and stirred for 15 min, the organic layer was separated, the aqueous phase was extracted once with ethyl acetate (725 L).

The combined organic phase was washed twice with 10% brine (725 L×1, 483 L×1), and then the organic phase was concentrated under reduced pressure at 45-55° C. MTBE (242 L) was added to the residue and stirred for 1 to 2 hours, then n-heptane (121 L) is added slowly over 2-3 hours at 15-25° C. The resulting slurry was filtered, the filter cake was washed once with MTBE (30 L). The wet cake was dried at 45-55° C. under vacuum for 6-10 h to afford compound (IX) as a yellow solid (74.70 kg, 72.7% yield).

HPLC Purity: 98.8% (a/a), RT=8.81 min.

¹H NMR: (400 MHz, CDCl₃) δ [ppm] 7.45-7.40 (m, 4H), 7.32-7.19 (m, 6H), 4.35 (s, 1H), 3.70-3.65 (m, 1H), 3.26-3.10 (m, 3H), 2.85 (s, 3H), 2.65-2.57 (m, 2H), 0.85-0.82 (d, 3H).

### 2.5 Preparation of (2R,3S)-2-methyl-3-((methylsulfonyl)methyl) azetidine hydrochloride (Ib)

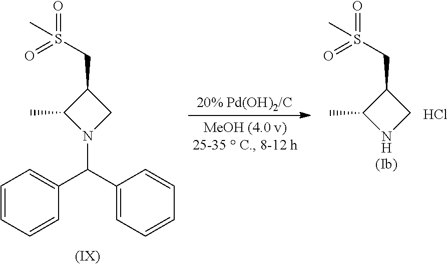

To a 1000 L reactor was charged compound (IX) (143.9 kg, 436.8 mol, 1.0 eq) and MeOH (447.50 kg), then 20% Pd(OH)₂/C (28.78 kg, 20% w/w %) and AcOH (26.21 kg, 436.8 mol, 1.0 eq) was added separately. The mixture was subjected to hydrogenolysis conditions under 0.5-1.0 MPa H₂ at 25-35° C. over 8-12 h. Upon reaction completion (HPLC monitoring), the reaction mixture was filtered, and the filter cake was washed once with MeOH (144 L). 4 M HCl/MeOH was added to the filtrate to adjust the pH to a target range of 1-2. The mixture was concentrated under vacuum at 45-55° C. to 400 L volume. The residue was washed twice with n-heptane (288 L×2), n-heptane phase was discarded. The residue was then concentrated under reduced pressure at 45-55° C. MeOH (144 L) was added to the residue and stirred for 0.5-1 h at 45-55° C., then THF (864 L) was added slowly to the mixture over 2-3 hours at 45-55° C. The mixture was cooled to 20-30° C. over 5 h and stirred for another 4-5 hours. The resulting slurry was filtered, and the filter cake was washed once with THF (32 L). The wet cake was dried at 45-55° C. under vacuum for 6-10 h to afford compound (Ib) as a white solid (76.20 kg, 87.7% yield).

GC Purity: 98.9% (a/a), RT=18.98 min.

¹H NMR: (400 MHz, DMSO-d₆) δ [ppm] 9.22-9.13 (m, 2H), 4.32-4.25 (m, 1H), 3.90 (m, 1H), 3.75 (m, 1H), 3.55 (m, 2H), 2.95 (m, 4H), 1.48-1.46 (d, 3H).

### Example 3a: Synthesis of 2-((3s,4R)-3-fluoro-4-methoxypiperidin-1-yl)pyrimidin-4-amine (Id)

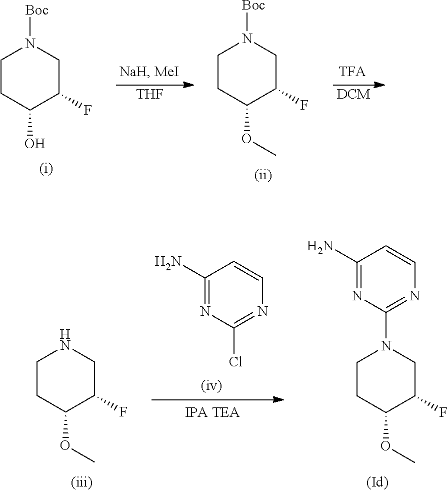

### 3.1 Synthesis of tert-butyl (3S,4R)-3-fluoro-4-hydroxypiperidine-1-carboxylate (i) Compound (i)

(tert-butyl (3S,4R)-3-fluoro-4-hydroxypiperidine-1-carboxylate was synthesized according to methods described in J. Org. Chem., 2013, 78, 8892-8897.

### 3.2 Synthesis of S (3S,4R)-tert-butyl 3-fluoro-4-methoxypiperidine-1-carboxylate (ii)

Sodium hydride (218.90 mg, 9.122 mmol, 4 equiv.) was added to (tert-butyl (3S,4R)-3-fluoro-4-hydroxypiperidine-1-carboxylate compound (i) (500 mg, 2.280 mmol, 1 equiv.) in THF (10 mL) at 0° C. After stirring for 20 min, methyl iodide (1294.73 mg, 9.122 mmol, 4 equiv.) was added. The resulting solution was stirred for additional 1 h at 0° C. The reaction was then quenched by addition of 10 mL of water. The solids were filtered out. The resulting solution was extracted with EA and concentrated under vacuum. This resulted in 500 mg (94.1%) of the title compound as light-yellow oil.

LC-MS: (ES, m/z)=178 [M+1-56].

### 3.3 Synthesis of (3S,4R)-3-fluoro-4-methoxypiperidine (iii)

The solution of tert-butyl (3S,4R)-3-fluoro-4-methoxypiperidine-1-carboxylate compound (ii) (500 mg, 2.143 mmol, 1 equiv.) in TFA/DCM (3/10 mL) was stirred for 1 h at rt. The resulting mixture was concentrated under vacuum to afford 500 mg (crude) of compound (iii) as a solid.

### 3.4 Synthesis of 2-((3S,4R)-3-fluoro-4-methoxypiperidin-1-yl)pyrimidin-4-amine (Id)

The mixture of (3S,4R)-3-fluoro-4-methoxypiperidine compound (iii) (3 g, 22.528 mmol, 1 equiv.), 2-chloropyrimidin-4-amine compound (iv) (2.33 g, 0.018 mmol, 0.8 equiv.) and TEA (6.84 g, 0.068 mmol, 3 equiv.) in IPA (3 mL) was stirred for 12 h at 100° C. The solvent was removed under vacuum and residue was purified by FLASH (5% MeOH in DCM) to give 3.3 g (66%) of compound (Id) as a light-yellow solid.

LC-MS: (ES, m/z)=227 [M+1].

¹H-NMR (400 MHz, 6d-DMSO) δ ppm 7.72 (d, 1H, J=5.6 Hz), 6.39 (s, 2H), 5.71 (d, 1H, J=5.6 Hz), 4.83 (d, 1H, J=49.3 Hz), 4.60-4.49 (m, 1H), 4.29 (d, 1H, J=13.3 Hz), 3.55-3.42 (m, 1H), 3.28 (d, 1H, J=13.3 Hz), 3.20-3.04 (m, 1H), 1.76-1.48 (m, 2H).

### Example 3b: Synthesis of 2-((3s,4R)-3-fluoro-4-methoxypiperidin-1-yl)pyrimidin-4-amine (Id)

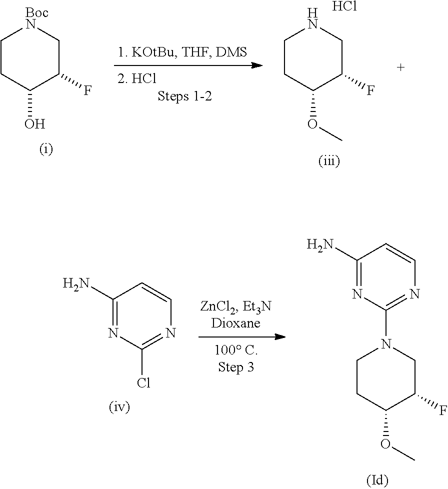

### Steps 1 and 2: Synthesis of (3S,4R)-3-fluoro-4-methoxypiperidine (iii)

Exactly 75 mL of tetrahydrofuran and 30.0 g (0.1368 mol) of N-Boc-(3S,4R)-3-fluoro-4-hydroxypiperidine compound (i) was charged to a 250 mL 3-necked round bottom flask fitted with an overhead stirrer and a nitrogen inlet/outlet. Then 5 g (6.4 mL) of tert-butyl alcohol was charged and the glass funnel was rinsed with 2.5 g (2.8 mL) of tetrahydrofuran (note: toluene/THF could also be used). To this was added 26 g (0.20 mol, 1.5 mol eq) of dimethyl sulfate (note: CH₃I could also be used) and the resulting mixture stirred for 5 min. Potassium-tert-butylate 20% in THF (26.5 g, 0.24 mol, 1.75 mol eq) was added via addition funnel over a 1 h period maintaining the internal temperature between 20-30° C. The addition was exothermic, and the temperature was controlled by the rate of addition. The reaction mixture thickened initially and then thinned out as the addition proceeds. After the addition was complete the addition funnel was rinsed with 3 mL of THF. The reaction mixture was stirred at 20-30° C. for 30 min and then sampled for reaction completion. The reaction was determined to be complete when <2.0%-a/a of compound (i) remains. The reaction can be held at 20-40° C. for 24 h without negatively impacting yield or quality. Then 30 mL of water was added to the reaction mixture with stirring.

A 2 L Erlenmeyer flask was charged with 850 ml of deionized water and 100 g of 25% ammonia solution and the resulting solution was stirred for 5 min. Then 32 mL of this solution was added to the reaction mixture followed by 15 mL of water maintaining the temperature between 20-30° C. The resulting mixture was stirred at this temperature for 2 h and then sampled for complete consumption of dimethyl sulfate. The dimethyl sulfate was determined to be completely quenched when <5 ppm remains. The mixture was transferred to a 250 mL separatory funnel and the layers allowed to separate for 30 min. The lower spent aqueous phase is drawn off for disposal. Any rag layer is combined with the organic layer. The upper product rich organic layer was allowed to stand for 5 min, then any additional spent aqueous layer is drawn off for disposal. The product-rich organic layer is transferred back into the 3-necked round bottom flask. Then 4.5 g of acetic acid was added to the mixture followed by 45 mL of water. The resulting biphasic mixture stirred at 20-30° C. for 30 min. Agitation was stopped and the biphasic mixture transferred back into the 250 mL separatory funnel. The layers were allowed to separate for 30 min, then the lower spent aqueous layer drawn off for disposal. The organic layer was allowed to stand for an additional 5 min and then any aqueous layer drawn off for disposal. The organic layer was transferred back into the 250 mL 3-necked flask and the mixture warmed to40-50° C. under a slight house vacuum until a gentle reflux is achieved and distill off about 20 mL of the THF/water azeotrope. Then 75 mL of toluene was added, the mixture warmed to 40-50° C. under a slight house vacuum until a gentle reflux was achieved and distill off about 20 mL of the THF/toluene/water azeotrope. The Step 1 mixture was sampled for Karl-Fischer analysis (KF). The KF endpoint is reached at <0.25%-w/w. The Step 1 mixture can be held for 72 h at 20-30° C. without negatively impacting yield or quality. The Step 1 mixture was transferred to a glass jar and 20 mL of toluene was added.

Exactly 80 g (100 mL) of isopropyl alcohol was charged to a 500 mL 3-necked flask fitted with an overhead stirrer and a nitrogen inlet/outlet. Via a gas inlet tube and with gentle stirring 25 g of hydrogen chloride (100%) was charged to the reactor making sure that the gas inlet tube was below the surface of the isopropyl alcohol. This addition was highly exothermic. The hydrochloric acid solution in toluene was stirred at 0-15° C. under nitrogen for 1 h. The temperature of the hydrochloric acid solution in isopropyl alcohol was adjusted to 20-30° C. and the Step 1 mixture was added dropwise over a 90 min period via an addition funnel. CO₂ off-gasing was observed, which was controlled by the rate of addition. After the addition was complete, the addition funnel was rinsed with 10 mL of toluene and the resulting slurry stirred at 20-30° C. for 3-4 h. The reaction was determined to be complete when <0.5%-a/a of Step 1 mixture remains. The slurry can be held at 20-30° C. for 24 h without negatively impacting yield or quality. The slurry was heated to 40-50° C. under slight vacuum until a gentle reflux is obtained and about 70-80 mL of isopropyl alcohol/toluene was distilled. An additional 100 mL of toluene was charged while distilling to maintain a constant volume in the 3-necked flask and about 100 mL of solvent was distilled off (repeated two times). The slurry was cooled to 20-30° C. and sampled to evaluate the solvent exchange. The endpoint is reached when <2%-a/a of isopropyl alcohol remained. 9 mL of isopropyl alcohol was charged to the slurry and stirred for an additional 1 h. The crystals were collected via filtration and the cake was washed with two cake volumes (about 50 mL) of toluene. The cake was deliquored for 1 h under nitrogen and then dried at 45-50° C. under vacuum for 24 h.

### Step 3. Synthesis of 2-((3S,4R)-3-fluoro-4-methoxypiperidin-1-yl)pyrimidin-4-amine (Id)

Exactly 80 g (78 mL) dioxane, 31.0 g (0.183 mol, 1.09 mol eq) compound (iii) 21.6 g (0.167 mol, 1.0 mol eq), 4-amino-2-chloropyrimidine, 14.5 g (0.027 mol, 0.16 mol eq), 25% ZnCl2 in 2-methyl tetrahydrofuran, and 44.0 g (60.0 mL, 0.43 mol) triethylamine was charged to a 500 mL 3-neccked flask fitted with an overhead stirrer and a nitrogen inlet/outlet. The resulting mixture was heated to reflux (90-100° C.) and stirred at this temperature for 16 h. The reaction mixture was cooled to 50-60° C. and sampled for reaction completion. The reaction was determined to be complete when <1.0%-a/a of 4-amino-2-chloropyrimidine remains. The reaction was cooled to 20-30° C. then 43 mL water and 110 g of 30% NaOH was added. The resulting biphasic mixture was stirred at 20-30° C. for 20 min. Agitation was stopped and the layers allowed to separate for 30 min. The lower spent aqueous phase was drawn off for disposal. Any rag layer is drawn off with the lower spent aqueous phase which was then sampled for pH determination. The pH of the spent aqueous layer was >12. The upper rich organic stream was allowed to settle for an additional 5 min. Any spent aqueous layer was drawn off for disposal. To the product rich organic layer was added 40 g of 30% NaOH and 16 g water. The resulting biphasic mixture was stirred at 20-30° C. for 20 min. Agitation was stopped and the layers allowed to separate for 30 min. The lower spent aqueous layer was drawn off for disposal. Any rag layer was drawn off and discarded. The upper product rich organic layer was allowed to settle for an additional 5 min. Any spent aqueous layer was drawn off for disposal. The product rich organic phase was polish filtered into a second 500 mL 3-necked flask fitted with an overhead stirrer and a nitrogen inlet/outlet. The first flask was rinsed with 1,4-dioxane (28 mL) and the rinse was transferred to the second flask. The solution was heated to 40-60° C. under house vacuum until a gentle reflux was achieved and 100-120 mL of 1,4-dioxane was distilled off. Then 130 mL of toluene was charged to the mixture and the resulting solution was warmed to 40-60° C. under house vacuum until a gentle reflux was achieved. The 1,4-dioxane/toluene solvent mixture was removed via distillation. An additional 200 mL of toluene was added during the distillation to maintain a constant volume. A total of 180-220 mL of 1,4-dioxane/toluene distillate was removed. The solvent exchange was determined to be complete when <5%-a/a of 1,4-dioxane remained. The resulting slurry was warmed to 65-75° C., stirred at this temperature for 30 min, cooled to 20-30° C. and then to 0-10° C. The slurry was held at this temperature for 30 min. The crystals were collected via filtration, washed with 30 mL of toluene and then deliquored under nitrogen for 30 min.

LC-MS: (ES, m/z)=227 [M+1].

¹H-NMR (400 MHz, 6d-DMSO) δ ppm 7.72 (d, 1H, J=5.6 Hz), 6.39 (s, 2H), 5.71 (d, 1H, J=5.6 Hz), 4.83 (d, 1H, J=49.3 Hz), 4.60-4.49 (m, 1H), 4.29 (d, 1H, J=13.3 Hz), 3.55-3.42 (m, 1H), 3.28 (d, 1H, J=13.3 Hz), 3.20-3.04 (m, 1H), 1.76-1.48 (m, 2H).

### Example 4a: Synthesis of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine (I)

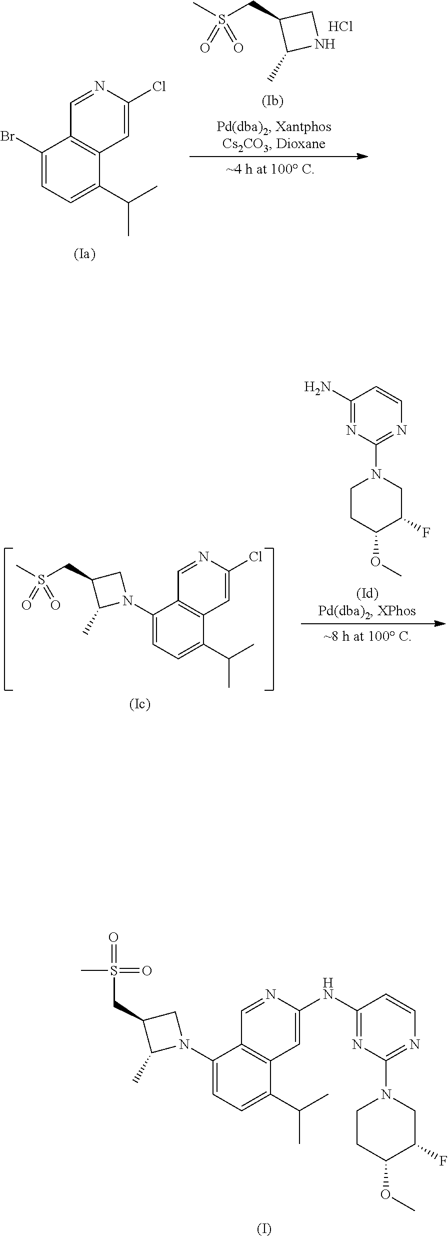

To a glass jacketed reactor (R1) at 20-30° C. was added 1,4-dioxane (18.0 L, 4.0 vol), compound (Ia) (6.0 kg, 1.0 eq), compound (Ib) (4.5 kg, 1.05 eq) and Cs₂CO₃ (23.6 kg, 3.4 eq). R1 was inerted with N₂ and vacuum (2 cycles), followed by charging Pd(dba)₂ (364 g, 0.03 eq) and XantPhos (366 g, 0.03 eq). R1 was inerted again with N₂ and vacuum (2 cycles), the batch heated to 100° C. for 4-8 hours, then cooled to 40-50° C. Then the reaction mixture containing compound (Ic) was cooled to 20-30° C. and used directly in the next step.

¹H NMR (CDCl₃): δ [ppm]=9.43 (s, 1H), 7.91 (s, 1H), 7.78 (d, 1H, 7.8 Hz), 7.45 (d, 1H, 7.8 Hz), 3.56 (hept, 1H, 6.9 Hz), 1.38 (d, 6H, 6.9 Hz).

To the reaction mixture containing compound (Ic) is added compound (Id) (4.2 kg, 1.05 eq), Pd(dba)₂ (352 g, 0.04 eq), XPhos (501 g, 0.06 eq) and 1,4-dioxane (5 L, 0.83 vol) as a rinse. The batch was heated to 100° C. for 4 hours, then cooled to 50° C. and additional Pd(dba)₂ (241 g, 0.024 eq) was added with 1,4-dioxane (1 L, 0.15 vol) as a rinse. The reaction was stirred at 100° C. for another 4 hours.

The batch was cooled to 50-60° C., diluted with water (12 L, 2 vol), stirred at 55-65° C. for 30 minutes and the aqueous layer was removed (keep at 50° C. during layer separation). Water (9 L, 1.50 vol) and 38% (w/w) NaHSO₃ (10.4 kg, 2.2 eq) were added, the batch was stirred at 55-65° C. for 2 hours, then diluted with 1,4-dioxane (72 L, 12 vol). The batch was azeotropically dried by distillation (40-50° C., 200 mbar) to remove 14 volumes of distillate. Azeotropic distillation was continued by adding additional 1,4-dioxane (72 L, 12 vol), followed by distillation to remove another 12 vol of distillate. The batch was checked for water content (water is NMT 1.0%) and if water content was high, an additional 1,4-dioxane charging and distillation was repeated. After reaching NMT 1.0% water, the batch was diluted with 1,4-dioxane (84 L, 14 vol) and stirred at 65-75° C. for NLT 1 hour. The batch was cooled to 25° C. and filtered (R1 to R2) to remove Pd-bisulfite precipitate. R1 is rinsed with 1,4-dioxane (5 L, 0.80 vol) and sent through the filter to R2. The filtrate was concentrated (50-60° C., 150 mbar) to remove 16.3 volumes (˜98 L) of distillate.

Previously synthesized compound (I) seed crystals (0.15% w/w) were added at 50-60° C., followed by slow addition of EtOH (60 L, 10 vol) anti-solvent at 50-60° C. over NLT 1 hour. The ratio of 1,4-dioxane to EtOH was checked (1,4-dioxane NMT 15%), and then the reaction was slowly cooled to 15-25° C. over NLT 3 hour and stirred for another NLT 3 hours. The resulting Compound (I) solids were isolated by filtration, displacement-washed with EtOH (9 L, 1.4 vol), followed by two re-slurry washes with water (2×18 L, 2×2.8 vol) and then with three EtOH displacement wash (3×6 L, 3×0.9 vol). Compound (I) was then dried under vacuum (50 mbar, 65-75° C.) to provide compound (I) Crude (5.6 kg, 37% yield, Purity by HPLC 94% a/a).

HPLC: 93.7% (a/a).

¹H NMR (DMSO-d₆): δ [ppm]=9.94 (1H, bs), 9.07 (1H, bs), 8.65 (1H, bs), 8.01 (1H, d, J=5.67), 7.42 (1H, d, J=8.08), 6.56 (1H, d, J=8.08), 6.49 (1H, d, J=5.67), 4.94 (1H, dddd, J=50.0, 4.95, 2.17, 2.17), 4.74 (1H, dddd, J=14.35, 9.53, 5.31, 1.57), 4.67 (1H, dd, J=7.25, 7.25), 4.49 (1H, bd, J=12.46), 4.20 (1H, dq, J=6.25, 6.25), 3.64 (1H, dd, J=7.25, 7.25), 3.59 (1H, dddd, J=24.88, 10.15, 4.44, 2.26), 3.57 (1H, dd, J=14.34, 6.33), 3.53-3.48 (1H, bm), 3.52-3.44 (1H, m), 3.51 (1H, dd, J=14.34, 8.33), 3.37 (3H, s), 3.29 (1H, ddd, J=12.46, 10.12, 3.13), 3.00 (3H, s), 2.90 (1H, dddd, J=7.80, 7.80, 7.80, 7.80), 1.82 (1H, dddd, J=12.95, 4.27, 4.27, 3.99), 1.75 (1H, ddddd, J=10.56, 10.56, 10.56, 4.22, 1.64), 1.43 (3H, d, J=6.09), 1.31 (3H, d, J=6.95), 1.30 (3H, d, J=6.92).

### Example 4b: Synthesis of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine (I)

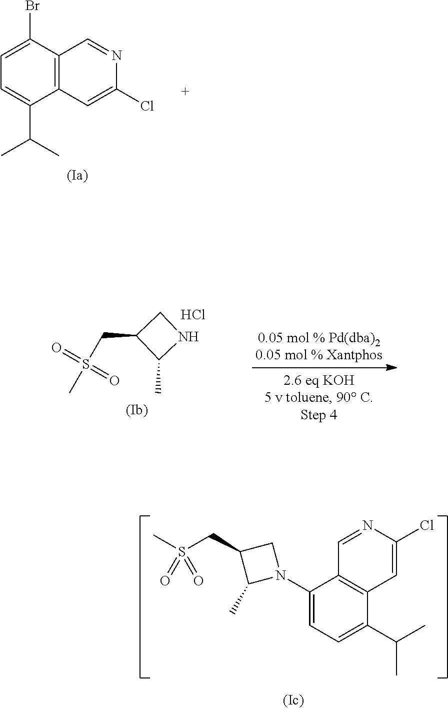

Exactly 44 g (45 mL) of toluene, 12.3 g (43.2 mmol, 1.0 eq) of 8-bromo-3-chloro-5-isoquinoline (compound (Ia)), 9.1 g (45.6 mmol, 1.05 mol eq) of (2R,3S)-2-methyl-3-((methylsulfonyl)methyl)azetidine HCl (compound (Ib)), 110 mg (0.2 mmol, 0.005 mol eq) Bis(dibenzylideneacetone)palladium(0) (Pd(dba)₂, 110 mg (0.2 mmol, 0.005 mol eq) Xantphos was charged under nitrogen to a 250 mL 3-necked flask fitted with an overhead stirrer and a reflux condenser. The flask was evacuated twice with house vacuum and then the vacuum broken with nitrogen. Then 11.8 g (7.7 mL, 105 mmol, 2.65 mol eq) of 50% potassium hydroxide and 8.0 mL of deionized water was added. The flask was evacuated again with house vacuum and the vacuum broken with nitrogen. The resulting biphasic mixture was warmed to 85-95° C. and held at this temperature for 10 h. The reaction mixture was cooled to 55-65° C. The reaction was determined to be complete when <1.0%-a/a of compound (Ia) remains. The reaction mixture was cooled to 20-30° C. and then charged with 9 mL of deionized water followed by 9 mL of toluene. The biphasic mixture was stirred for an additional 0.5 h. The agitation was stopped, and the mixture was transferred to a separatory funnel and the layers were allowed to separate for 0.5 h. The lower spent aqueous stream along with any rag layer were disposed. The upper product rich organic phase stood for 5 min, and the aqueous layer was disposed. The product rich organic stream was charged back into the 3-necked flask. 9 mL of deionized water and 30 mg (30 uL) of acetic acid was charged into a separate 25 mL Erlenmeyer flask. The aqueous acetic acid was charged to the organic layer and the biphasic mixture was stirred for 30 min. The agitation was stopped, the biphasic mixture was transferred into to a separatory funnel and the layers were allowed to separate for 0.5 h. The lower spent aqueous stream along with any rag layer was disposed. The product rich organic stream stood for 5 min, any aqueous layer was disposed. The product rich toluene stream was transferred back into the 3-necked flask, heated to reflux to distill off 15-25 mL of the toluene/water azeotrope. An additional 40 mL of toluene was charged to the 3-necked flask, which was warmed to reflux to distill off an additional 40 mL of the toluene/water azeotrope. The product rich toluene stream was cooled to 20-30° C. and sampled for KF. When the KF was <0.2%-w/w, the mixture (compound Ic) was polish filtered through a celite pad, the pad was rinsed with about 2 mL of toluene and mixed well.

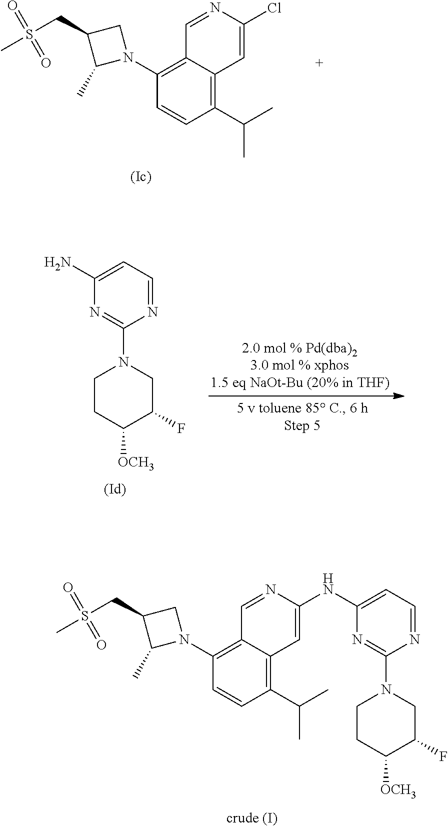

Exactly 42 mL of the compound Ic solution prepared above containing 15.06 g (0.041 mol, 1.03 eq) (assay 35.95%) was charged to a 250 mL 3-necked round bottom flask fitted with an overhead stirrer and a nitrogen inlet/outlet. Then 9.0 g (0.0398 mol, 1.00 eq) of compound (Id), 46 mg (0.08 mmol, 0.02 mol eq) of bis(dibenzylideneacetone)palladium, 39 mg (0.08 mmol, 0.02 mol eq) of Xphos and 7.5 mL of toluene was charged to the flask. The reactor was evacuated twice with house vacuum. The vacuum was broken with nitrogen. Then 29 g (0.06 mol, 1.5 mol eq) of a solution of 20 wt % sodium tert-butylate in THF was charged to the flask followed by 3 mL of toluene. The resulting mixture was warmed to 45° C. and held for 15 min, then warmed slowly to 80-95° C. The reaction mixture was held at this temperature for 6 h. The mixture was cooled to 55-60° C. The reaction was determined to be complete when <4%-a/a of either compound (Ic) or compound (Id) remains. The reaction was charged 63 mL of 1,4-dioxane followed by 3.5 mL (3.67 g, 0.061 mol) of acetic acid.

In a separate 500 mL Erlenmeyer flask was charged 23 g (0.141 mol) of N-acetyl cysteine, 284 mL of deionized water and 20 mL of 30 wt % sodium hydroxide. The mixture was stirred at room temperature for 15 min. 25 mL of this solution was charged to the reaction mixture. The mixture was heated to 55-60° C. and stirred at this temperature for 30 min. Agitation was stopped, the mixture was transferred to a 250 mL separatory funnel and the layers were allowed to separate for 30 min. The lower dark brown spent aqueous layer was disposed. The rag layer was combined with the upper product rich organic phase, and the upper organic phase was allowed to stand for 15 min. The additional spent aqueous layer was disposed. The reaction mixture was transferred back into the 3-necked round bottom flask and charged with 25 mL of the N-acetyl cysteine solution. The resulting biphasic mixture was warmed to 55-60° C. and stirred at this temperature for 30 min. Agitation was stopped, the mixture was transferred to a 250 mL separatory funnel and the layers were allowed to separate for 30 min. The lower dark brown spent aqueous layer was disposed. The rag layer was combined with the upper product rich organic phase, and the upper organic phase was allowed to stand for 15 min. The additional spent aqueous layer was disposed. The organic phase was transferred back into the 3-necked round bottom flask and charged with 25 mL of the N-acetyl cysteine solution. The resulting biphasic mixture was warmed to 55-60° C. and stirred at this temperature for 30 min. Agitation was stopped, the mixture was transferred to a 250 mL separatory funnel and the layers were allowed to separate for 30 min. The lower dark brown spent aqueous layer was disposed. The rag layer was combined with the upper product rich organic phase, and the upper organic phase was allowed to stand for 15 min. The additional spent aqueous layer was disposed. 27 mL of deionized water was charged to the reaction mixture. 10 g of 1,4-dioxane (peroxide free) was charged to the reaction mixture, which was heated to 55-60° C. and held at this temperature for 30 min. Agitation was stopped, the mixture was transferred to a separatory funnel and the layers were allowed to separate for 30 min. The lower dark brown spent aqueous layer was disposed. The rag layer was combined with the upper product rich organic phase, and the upper organic phase was allowed to stand for 15 min. The additional spent aqueous layer was disposed. The reaction mixture was charged back into the 3-necked round bottom flask and 50 mL of toluene was charged to the mixture. The mixture was heated to reflux (80-120° C.) and 50 mL of toluene/dioxane/water azeotrope was distilled. An additional 90-110 mL of toluene was added to the mixture to maintain a constant volume during the distillation. The product rich toluene stream was cooled to 70-90° C. and checked for crystallization. The crystal slurry was cooled to 15-25° C. over a 4 h period maintaining an inert atmosphere with nitrogen. The crystal slurry was stirred at this temperature for an additional 2 h. The crystals were collected via filtration. The cake was washed via displacement wash with about 30 mL of toluene. The filter cake was washed twice with about 30 mL of ethanol denatured with toluene and the cake was deliquored to provide crude compound (I).

LC-MS: (ES, m/z)=557 [M+1].

¹H NMR (DMSO-d₆): δ [ppm]=9.94 (1H, bs), 9.07 (1H, bs), 8.65 (1H, bs), 8.01 (1H, d, J=5.67), 7.42 (1H, d, J=8.08), 6.56 (1H, d, J=8.08), 6.49 (1H, d, J=5.67), 4.94 (1H, dddd, J=50.0, 4.95, 2.17, 2.17), 4.74 (1H, dddd, J=14.35, 9.53, 5.31, 1.57), 4.67 (1H, dd, J=7.25, 7.25), 4.49 (1H, bd, J=12.46), 4.20 (1H, dq, J=6.25, 6.25), 3.64 (1H, dd, J=7.25, 7.25), 3.59 (1H, dddd, J=24.88, 10.15, 4.44, 2.26), 3.57 (1H, dd, J=14.34, 6.33), 3.53-3.48 (1H, bm), 3.52-3.44 (1H, m), 3.51 (1H, dd, J=14.34, 8.33), 3.37 (3H, s), 3.29 (1H, ddd, J=12.46, 10.12, 3.13), 3.00 (3H, s), 2.90 (1H, dddd, J=7.80, 7.80, 7.80, 7.80), 1.82 (1H, dddd, J=12.95, 4.27, 4.27, 3.99), 1.75 (1H, ddddd, J=10.56, 10.56, 10.56, 4.22, 1.64), 1.43 (3H, d, J=6.09), 1.31 (3H, d, J=6.95), 1.30 (3H, d, J=6.92).

### Example 5a: Recrystallization of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine (I)

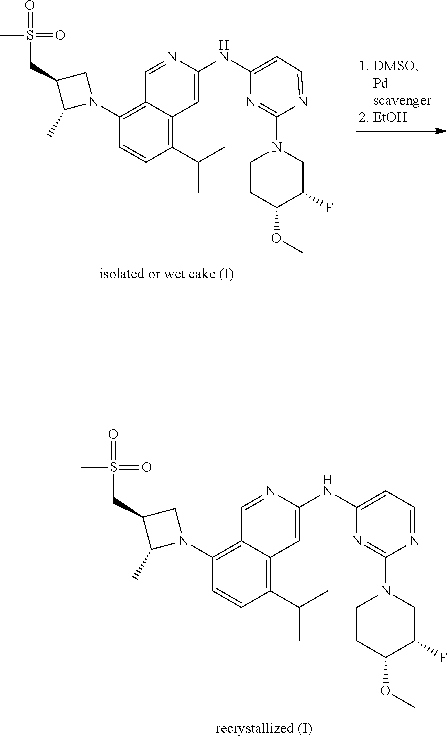 [SMILES: CO[C@@H]1CCN(c2nccc(Nc3cc4c(C(C)C)ccc(N5C[C@H](CS(C)(=O)=O)[C@H]5C)c4cn3)n2)C[C@@H]1F ; CO[C@@H]1CCN(c2nccc(Nc3cc4c(C(C)C)ccc(N5C[C@H](CS(C)(=O)=O)[C@H]5C)c4cn3)n2)C[C@@H]1F]

Crude compound (I) (6.3 kg) obtained from Example 4 was dissolved in DMSO (10 L, 1.5 vol) and treated with a Pd scavenger Quadrasil MP (850 g, 14% w/w equivalents based on compound (I)). After stirring at 70° C. for 1.5 hours, the scavenger was removed by filtration (50° C.), the silica gel scavenger solids were washed with DMSO (5 L, 0.8 vol), and all filtrates were combined. The batch was heated to 55° C. and EtOH (95 L, 15.0 vol) was added slowly over NLT 3 hour (the mixture was seeded with previously synthesized compound (I) after 2 vol of EtOH were added), then the batch was cooled to 45° C. Cooling continued slowly to 10° C. with stirring for NLT 2 hours. The batch was filtered and the compound (I) solids were washed twice with EtOH displacement washes (3×6.3 L, 3×1.0 vol). The compound (I) solid was dried under vacuum (50° C., 35 mbar) for NLT 16 hours to provide recrystallized compound (I) (4.9 kg, 78% yield, Purity by HPLC 99.3% a/a)

HPLC: 99.3% (a/a)

¹H NMR (DMSO-d₆): δ [ppm]=9.94 (1H, bs), 9.07 (1H, bs), 8.65 (1H, bs), 8.01 (1H, d, J=5.67), 7.42 (1H, d, J=8.08), 6.56 (1H, d, J=8.08), 6.49 (1H, d, J=5.67), 4.94 (1H, dddd, J=50.0, 4.95, 2.17, 2.17), 4.74 (1H, dddd, J=14.35, 9.53, 5.31, 1.57), 4.67 (1H, dd, J=7.25, 7.25), 4.49 (1H, bd, J=12.46), 4.20 (1H, dq, J=6.25, 6.25), 3.64 (1H, dd, J=7.25, 7.25), 3.59 (1H, dddd, J=24.88, 10.15, 4.44, 2.26), 3.57 (1H, dd, J=14.34, 6.33), 3.53-3.48 (1H, bm), 3.52-3.44 (1H, m), 3.51 (1H, dd, J=14.34, 8.33), 3.37 (3H, s), 3.29 (1H, ddd, J=12.46, 10.12, 3.13), 3.00 (3H, s), 2.90 (1H, dddd, J=7.80, 7.80, 7.80, 7.80), 1.82 (1H, dddd, J=12.95, 4.27, 4.27, 3.99), 1.75 (1H, ddddd, J=10.56, 10.56, 10.56, 4.22, 1.64), 1.43 (3H, d, J=6.09), 1.31 (3H, d, J=6.95), 1.30 (3H, d, J=6.92).

### Example 5b: Recrystallization of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine (I)

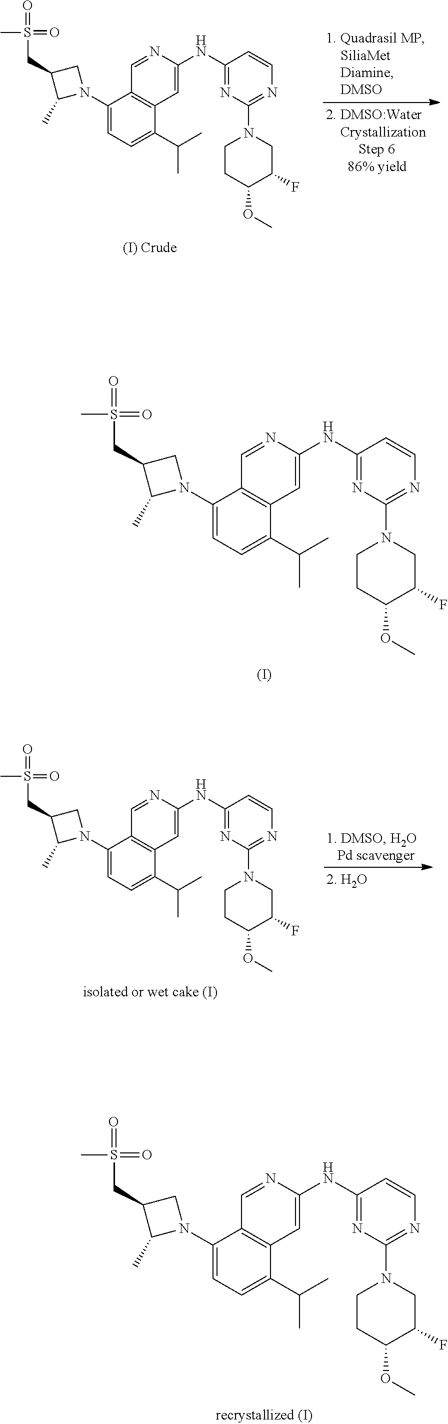 [SMILES: CO[C@@H]1CCN(c2nccc(Nc3cc4c(C(C)C)ccc(N5C[C@H](CS(C)(=O)=O)[C@H]5C)c4cn3)n2)C[C@@H]1F ; CO[C@@H]1CCN(c2nccc(Nc3cc4c(C(C)C)ccc(N5C[C@H](CS(C)(=O)=O)[C@H]5C)c4cn3)n2)C[C@@H]1F ; "(I)" ; CO[C@@H]1CCN(c2nccc(Nc3cc4c(C(C)C)ccc(N5C[C@H](CS(C)(=O)=O)[C@H]5C)c4cn3)n2)C[C@@H]1F ; CO[C@@H]1CCN(c2nccc(Nc3cc4c(C(C)C)ccc(N5C[C@H](CS(C)(=O)=O)[C@H]5C)c4cn3)n2)C[C@@H]1F]

To a 500 ml Jacketed 3-necked flask fitted with an overhead stirrer, a nitrogen inlet/outlet and a bottom valve was charged 65 mL of dimethylsulfoxide (DMSO), 5.4 g of Quadrasil MP (scavenger), 2.8 g SiliaMetS Diamine and 23 g (0.0413 mol) of compound I. The resulting suspension warmed to 85-95° C. and held at this temperature for 2 h. The suspension was cooled to 65-75° C. and filtered. The spent Quadrasil/SiliaMetS Diamine cake was washed with 25 mL of hot (65-75° C.) DMSO. The combined filtrate and wash was heated to 85-95° C. Then 23 mL of deionized water was added slowly over a 10 min period followed by the addition of 2 g of seed crystals. Finally, 8 mL of water was added over a 20 min period. The resulting slurry was stirred at 85-95° C. for 2 h, then cooled to 15-25° C. over a 4 h period and held at this temperature for at least 3 h. The crystals were collected via filtration, washed with 60 mL of absolute ethanol, deliquored for 1 h under nitrogen and dried under vacuum at 50-55° C. for 24 h.

LC-MS: (ES, m/z)=557 [M+1].

¹H NMR (DMSO-d₆): δ [ppm]=9.94 (1H, bs), 9.07 (1H, bs), 8.65 (1H, bs), 8.01 (1H, d, J=5.67), 7.42 (1H, d, J=8.08), 6.56 (1H, d, J=8.08), 6.49 (1H, d, J=5.67), 4.94 (1H, dddd, J=50.0, 4.95, 2.17, 2.17), 4.74 (1H, dddd, J=14.35, 9.53, 5.31, 1.57), 4.67 (1H, dd, J=7.25, 7.25), 4.49 (1H, bd, J=12.46), 4.20 (1H, dq, J=6.25, 6.25), 3.64 (1H, dd, J=7.25, 7.25), 3.59 (1H, dddd, J=24.88, 10.15, 4.44, 2.26), 3.57 (1H, dd, J=14.34, 6.33), 3.53-3.48 (1H, bm), 3.52-3.44 (1H, m), 3.51 (1H, dd, J=14.34, 8.33), 3.37 (3H, s), 3.29 (1H, ddd, J=12.46, 10.12, 3.13), 3.00 (3H, s), 2.90 (1H, dddd, J=7.80, 7.80, 7.80, 7.80), 1.82 (1H, dddd, J=12.95, 4.27, 4.27, 3.99), 1.75 (1H, ddddd, J=10.56, 10.56, 10.56, 4.22, 1.64), 1.43 (3H, d, J=6.09), 1.31 (3H, d, J=6.95), 1.30 (3H, d, J=6.92).

What is claimed is:

1. A method of preparing a compound of formula (Ic): 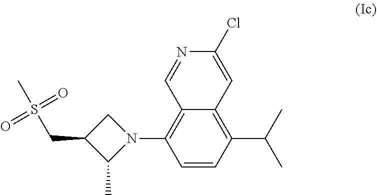 [SMILES: CC(C)c1ccc(N2C[C@H](CS(C)(=O)=O)[C@H]2C)c2cnc(Cl)cc12]
comprising reacting a first starting material of formula (Ia); 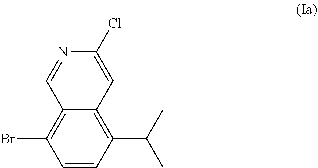
or a salt thereof,
with a second starting material of formula (Ib): 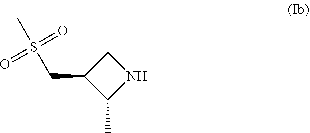
or a salt thereof,
in the presence of a base, a palladium catalyst, and a phosphine ligand to form the compound of formula (Ic).

2. The method of claim 1, wherein the base is cesium carbonate (Cs₂CO₃), the palladium catalyst is bis(dibenzylideneacetone)palladium(0) (Pd(dba)₂), and the phosphine ligand is (9,9-dimethyl-9H-xanthene-4,5-diyl)bis(diphenylphosphane) (Xantphos).

3. The method of claim 1, wherein the base is potassium hydroxide (KOH), the palladium catalyst is bis(dibenzylideneacetone)palladium(0) (Pd(dba)₂), and the phosphine ligand is (9,9-dimethyl-9H-xanthene-4,5-diyl)bis(diphenylphosphane) (Xantphos).

4. The method of any one of claims 1-3, wherein the reaction is carried out in dioxane at 90° C. to 110° C.

5. A method of preparing a compound of formula (III): 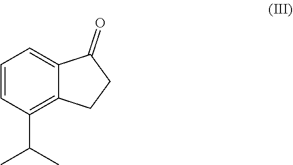
comprising hydrogenating a starting material of formula (II): 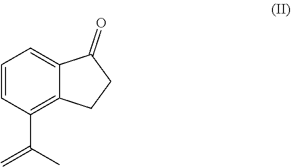
in the presence of a platinum hydrogenolysis catalyst or a palladium hydrogenolysis catalyst to form the compound of formula (III).

6. The method of claim 5, wherein the platinum hydrogenolysis catalyst is PtO₂ and the palladium hydrogenolysis catalyst is wet palladium/carbon.

7. The method of claim 5 or claim 6, wherein the reaction is carried out in ethyl acetate (EtOAc) at 20° C. to 30° C.

8. A method of preparing a compound of formula (IV): 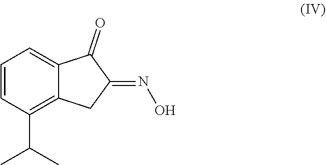
comprising reacting a starting material of formula (III): 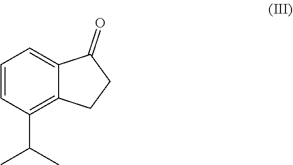
with tert-butyl nitrite (t-BuONO) and hydrogen chloride to form the compound of formula (IV).

9. The method of claim 8, wherein the reaction is carried out in tetrahydrofuran (THF) at 0° C. to 10° C., and wherein the hydrogen chloride is methanolic hydrogen chloride.

10. A method of preparing a compound of formula (V): 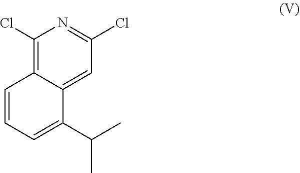
or a salt thereof, comprising reacting a starting material of formula (IV): 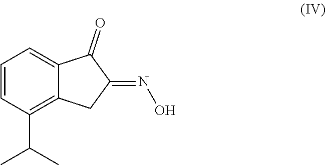
or a salt thereof with phosphoryl chloride (POCl₃), phosphorus pentachloride (PCl₅), and hydrogen chloride to form the compound of formula (V).

11. The method of claim 10, wherein the starting material is combined with POCl₃ and PCl₅ at 0° C. to 25° C., followed by addition of hydrogen chloride and warming to 50° C. to 70° C., and wherein the reaction is carried out in dioxane.

12. A method of preparing a compound of formula (VI): 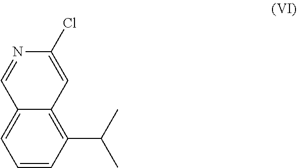
comprising reacting a starting material of formula (V): 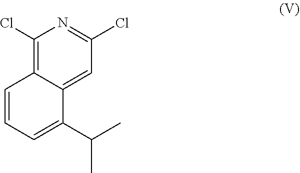
or a salt thereof in the presence of an amine base, a hydride reducing agent, and a palladium catalyst to form the compound of formula (VI).

13. The method of claim 12, wherein the palladium catalyst is 1,1′-Bis(diphenylphosphino)ferrocene]dichloropalladium(II) (Pd(dba)₂), the hydride reducing agent is sodium borohydride (NaBH₄) and the amine base is tetramethylethylenediamine (TMEDA).

14. The method of claim 12 or claim 13, wherein the reaction is carried out in tetrahydrofuran (THF) at 20° C. to 30° C.

15. A method of preparing a compound of formula (Ia): 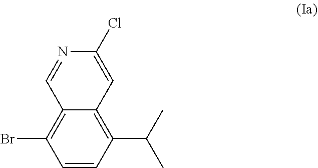
comprising reacting a starting material of formula (VI): 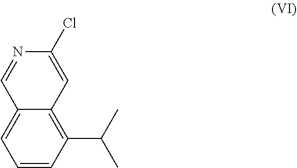
with a brominating agent in an acid to form the compound of formula (Ia).

16. The method of claim 15, wherein the brominating agent is N-bromosuccinimide (NBS) and the acid is sulfuric acid (H₂SO₄).

17. A compound selected from the following: 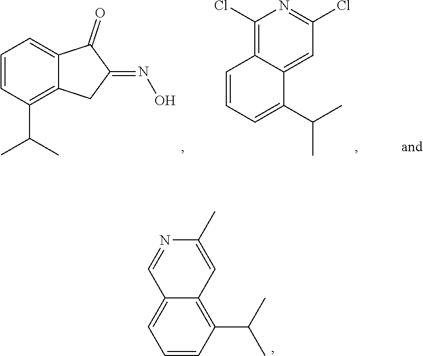
or a salt thereof.

18. A method of preparing a compound of formula (VII): 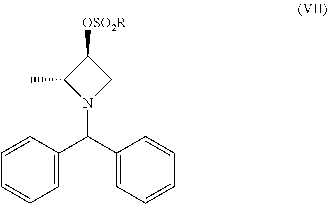
comprising reacting a starting material of formula (VIIa): 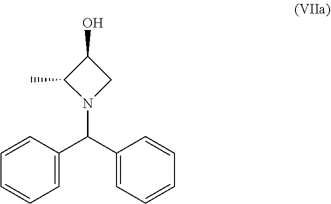 [SMILES: C[C@@H]1[C@@H](O)CN1C(c1ccccc1)c1ccccc1]
with a sulfonyl chloride (RSO₂Cl) and an amine base, e.g., ethanesulfonyl chloride (EsCl) and triethylamine (TEA), to form the compound of formula (VII), wherein R is a C₁-C₄ straight or branched alkyl group, or a phenyl group optionally substituted with halogen, a C₁-C₄ alkyl group, and/or a nitro group.

19. The method of claim 18, wherein the reaction is carried out in dichloromethane at 5° C. to 20° C.

20. A compound of formula (VIIc): 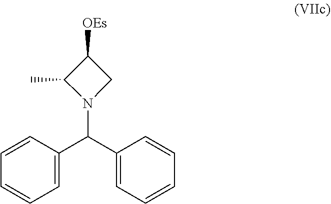
or a salt thereof, wherein Es is ethyl sulfonyl.

21. A method of preparing a compound of formula (VIII): 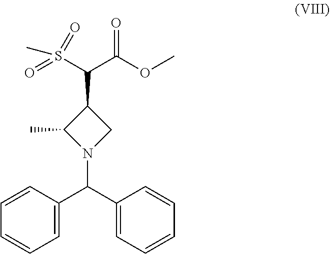 [SMILES: COC(=O)C([C@H]1CN(C(c2ccccc2)c2ccccc2)[C@@H]1C)S(C)(=O)=O]
comprising reacting a first starting material of formula (VIIc): 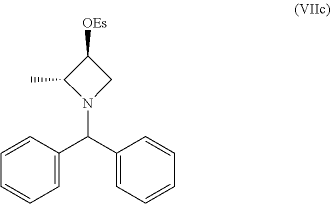
or a salt thereof with a second starting material of formula (VIIIb): 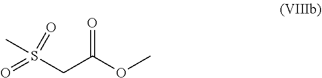
and potassium carbonate (K₂CO₃) to form the compound of formula (VIII).

22. The method of claim 21, wherein the second starting material of formula (VIIIb) is prepared by reacting methyl 2-bromoacetate with

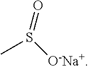

23. A method of preparing a compound of formula (IX): 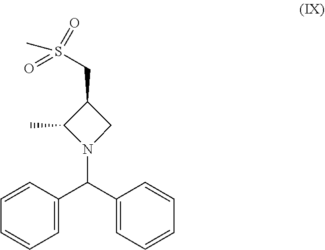 [SMILES: C[C@@H]1[C@@H](CS(C)(=O)=O)CN1C(c1ccccc1)c1ccccc1]
comprising reacting a starting material of formula (VIII): 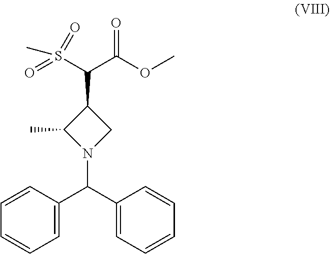 [SMILES: COC(=O)C([C@H]1CN(C(c2ccccc2)c2ccccc2)[C@@H]1C)S(C)(=O)=O]
or a salt thereof with lithium chloride (LiCl) to form the compound of formula (IX).

24. The method of claim 23, wherein the reaction is carried out in dimethylacetamide (DMAc) at 160° C. to 170° C.

25. A method of preparing a compound of formula (Ib): 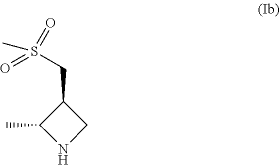
or a salt thereof comprising hydrogenating a starting material of formula (IX): 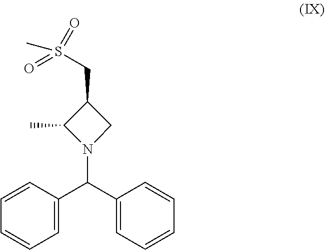 [SMILES: C[C@@H]1[C@@H](CS(C)(=O)=O)CN1C(c1ccccc1)c1ccccc1]
or a salt thereof in the presence of a palladium hydrogenolysis catalyst to form the compound of formula (Ib).

26. The method of claim 25, wherein the palladium hydrogenolysis catalyst is palladium hydroxide on carbon, 20 wt. % dry basis (20% Pd(OH)₂/C) and wherein the reaction is carried out in methanol (MeOH) at 30° C. to 50° C.

27. A method of preparing a compound of formula (I): 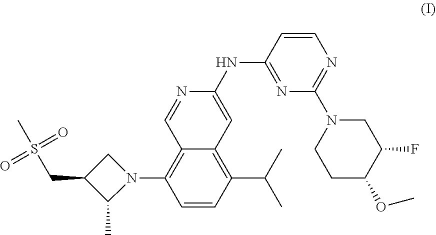 [SMILES: CO[C@@H]1CCN(c2nccc(Nc3cc4c(C(C)C)ccc(N5C[C@H](CS(C)(=O)=O)[C@H]5C)c4cn3)n2)C[C@@H]1F]
comprising reacting a first starting material of formula (Ic) or a salt thereof: 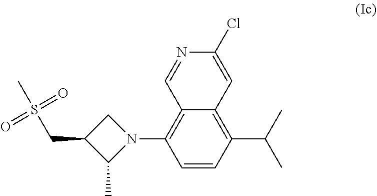 [SMILES: CC(C)c1ccc(N2C[C@H](CS(C)(=O)=O)[C@H]2C)c2cnc(Cl)cc12]
with a second starting material of formula (Id) or a salt thereof: 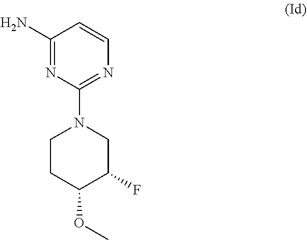 [SMILES: CO[C@@H]1CCN(c2nccc(N)n2)C[C@@H]1F]
in the presence of a palladium catalyst and a phosphine ligand to form the compound of formula (I), wherein the palladium catalyst and the phosphine ligand are separate compounds or a complex comprising both the palladium catalyst and the phosphine ligand.

28. The method of claim 27, wherein the palladium catalyst and phosphine ligand is other than the complex methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(II) (BrettPhos-Pd-G4).

29. The method of claim 27 or claim 28, wherein the palladium catalyst is selected from the group consisting of Pd(dppe)₂ (Bis[1,2-bis(diphenylphosphino)ethane]palladium(0)), CX-11 (1,3-Bis(2,6-diisopropylphenyl)imidazol-2-ylidene(1,4-naphthoquinone)palladium(0) dimer), CX-12 (1,3-Bis(2,4,6-trimethylphenyl)imidazol-2-ylidene (1,4-naphthoquinone)palladium(0) dimer), Pd(t-Bu₃P)₂ (Bis(tri-tert-butylphosphine)palladium(0)), Pd(PCy₃)₂ (Bis(tricyclohexylphosphine)palladium(0)), Pd(PPh₃)₄(Tetrakis(triphenylphosphine)palladium(0)), Pd₂(dba)₃ (Tris(dibenzylideneacetone)dipalladium(0)), Pd(OAc)₂ (Palladium (II) acetate), PdCl₂(PPh₃)₂(Dichlorobis(triphenylphosphine)palladium(II)), PdCl₂(Amphos)₂ (Bis(di-tert-butyl(4-dimethylaminophenyl)phosphine)dichloropalladium(II)), Pd(MeCN)₂Cl₂ (Bis(acetonitrile)dichloropalladium(II)), PdCl₂(P(o-Tol)₃)₂ (Dichlorobis(tri-o-tolylphosphine)palladium(II)), Pd(dppf)Cl₂ (1,1′-Bis(diphenylphosphino)ferrocene]dichloropalladium(II)), Pd(MeCN)₄(BF₄)₂ (Tetrakis(acetonitrile)palladium(II) tetrafluoroborate), Pd-PEPPSI-IPent (Dichloro[1,3-bis(2,6-Di-3-pentylphenyl)imidazol-2-ylidene](3-chloropyridyl)palladium(II)), Pd-PEPPSI-IPr ([1,3-Bis(2,6-Diisopropylphenyl)imidazol-2-ylidene](3-chloropyridyl)palladium(II) dichloride), Pd-PEPPSI-SIPr ((1,3-Bis(2,6-Diisopropylphenyl)imidazolidene) (3-chloropyridyl) palladium(II) dichloride), and bis(dibenzylideneacetone)palladium(0) (Pd(dba)₂).

30. The method of any one of claims 27-29, wherein the phosphine ligand or the complex comprising the palladium catalyst and phosphine ligand is selected from the group consisting of Triphenyl phosphine(PPh3); Bis(tri-o-tolylphosphine) (P(o-Tol)₃)₂; Tri-tert-butoxy phosphine (Pt-Bu₃); Tri-tert-butylphosphonium tetrafluoroborate (Pt-Bu3HBF₄); Bis(tricyclohexylphosphine (PCy₃); Bis (1-adamanyl)butylphosphane (n-BuP(AD)₂); 2,2′-Bis(diphenylphosphino)-1,1′-binaphthyl (BINAP), (9,9-Dimethyl-9H-xanthene-4,5-diyl)bis(diphenylphosphane)(Xantphos), Bis[(2-diphenylphosphino)phenyl] ether (DPEPhos); 1,1′-Bis(diphenylphosphino)ferrocene (dppf); 1,1′-Bis(di-tert-butylphosphino)ferrocene (dcypf), 1,3-Bis(diphenylphosphino)propane (DPPP), (2-Biphenylyl)di-tert-butylphosphine (JohnPhos), Chloro(2-dicyclohexylphosphino-1,1′-biphenyl)[2-(2′-amino-1,1′-biphenyl)] (CyJohnPhos), 2-Dicyclohexylphosphino-2′-(N,N-dimethylamino)biphenyl (DavePhos), (2-Dicyclohexylphosphino-2′,6′-diisopropoxy-1,1′-biphenyl)[2-(2′-amino-1,1′-biphenyl)] (RuPhos), 2-Dicyclohexylphosphino-2′,6′-dimethoxybiphenyl (SPhos), [(2-Di-cyclohexylphosphino-3,6-dimethoxy-2′,4′,6′-triisopropyl-1,1′-biphenyl)-2-(2′-amino-1,1′-biphenyl)] (BrettPhos), 1,1′-Bis(di-tert-butylphosphino)ferrocene (dtbpf), 2-Di-tert-butylphosphino-2′,4′,6′-triisopropylbiphenyl (t-BuXPhos), [(2-Di-tert-butylphosphino-3,6-dimethoxy-2′,4′,6′-triisopropyl-1,1′-biphenyl)-2-(2′-amino-1,1′-biphenyl)] (t-BuBrettPhos), 2-Di-tert-butylphosphino-3,4,5,6-tetramethyl-2′,4′,6′-triisopropyl-1,1′-biphenyl (Me₄₋ₜBuXPhos), 5-(Di-tert-butylphosphino)-1′,3′,5′-triphenyl-1′H-1,4′bipyrazole (BippyPhos), Di(1-adamantyl)-2-morpholinophenylphosphine (MorDalPhos), palladium/1,3-bis-(2,6-diisopropylphenyl)imidazolinium chloride (IPrHCL), [2-(Di-1-adamantylphosphino)-2′,4′,6′-triisopropyl-3,6-dimethoxybiphenyl][2-(2′-amino-1,1′-biphenyl)]palladium(II) methanesulfonate (AdBrettPhos), (2-Dicyclohexylphosphino-2′,6′-diisopropoxy-1,1′-biphenyl)[2-(2′-amino-1,1′-biphenyl)]palladium(II) methanesulfonate (RuPhos), [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate (BrettPhos), [(2-{Bis[3,5-bis(trifluoromethyl)phenyl]phosphine}-3,6-dimethoxy-2′,4′,6′-triisopropyl-1,1′-biphenyl)-2-(2′-amino-1,1′-biphenyl)]palladium(II) methanesulfonate (JackiePhos), [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate (t-BuBrettPhos), Mesyl(2-(di-tert-butylphosphino)-1,1′-binaphthyl)[2-(2′-amino-1,1′-biphenyl)]palladium (TrixiePhos), (2-Biphenyl)di-tert-butylphosphine, 2′-(Di-tert-butylphosphino)-N,N-dimethylbiphenyl-2-amine (t-BuDavePhos), 2-Di-tert-butylphosphino-2′-methylbiphenyl (t-BuMePhos), Chloro(2-dicyclohexylphosphino-1,1′-biphenyl)[2-(2′ amino-1,1′-biphenyl)]palladium(II) (CyJohnPhos), 2-Dicyclohexylphosphino-2′-methylbiphenyl (MePhos), 2-Dicyclohexylphosphino-2′-(N,N-dimethylamino)biphenyl (PhDavePhos), 2-Dicyclohexylphosphino-2′-methoxy-4′,6′-di-tert-butylbiphenyl (VPhos), 2-[(tert-Butyl)phenylphosphino]-2′,6′-bis(N,N-dimethylamino)biphenyl, (PhCPhos), [(2-Dicyclohexylphosphino-2′,6′-bis(N,N-dimethylamino)-1,1′-biphenyl)-2-(2′-amino-1,1′-biphenyl)] palladium(II) methanesulfonate (CPhos), Methanesulfonato[2-diethylphosphino-2′,6′-bis(dimethylamino)-1,1-biphenyl](2′-amino-1,1′-biphenyl-2-yl)palladium(II) (EtCPhos), 2-Di(tert-butyl)phosphino-2′,4′,6′-triisopropyl-3-methoxy-6-methylbiphenyl (RockPhos), Di-1-adamantyl(4″-butyl-2″,3″,5″,6″-tetrafluoro-2′,4′,6′-triisopropyl-2-methoxy-meta-terphenyl)phosphine (AlPhos), 2-(t-Butylphenylphosphino)-2′,6′-dimethylamino-1,1′-biphenyl, ((t-Bu)PhCPhos), and dicyclohexyl[2′,4′,6′-tris(propan-2-yl)[1,1′-biphenyl]-2-yl]phosphane (XPhos).

31. The method of any of claims 27-30, wherein the palladium catalyst is bis(dibenzylideneacetone)palladium(0) (Pd(dba)₂).

32. The method of any of claims 27-31, wherein the phosphine ligand is dicyclohexyl[2′,4′,6′-tris(propan-2-yl)[1,1′-biphenyl]-2-yl]phosphane (XPhos).

33. The method of any one of claims 27-32, wherein the reaction mixture further comprises a base.

34. The method of claim 33, wherein the base is selected from the group consisting of potassium carbonate (K₂CO₃), cesium carbonate (Cs₂CO₃), potassium hydroxide (KOH), and sodium tert-butoxide (NaOtBu).

35. The method of claim 33 or claim 34, wherein the base is cesium carbonate (Cs₂CO₃) or sodium tert-butoxide (NaOtBu).

36. The method of any one of claims 27-35, wherein the reaction is carried out in a solvent selected from toluene, 1,4-dioxane, tetrahydrofuran (THF), methyl tetrahydrofuran (Me-THF), anisole, water (H₂O), or mixtures thereof.

37. The method of any one of claims 27-36, wherein the reaction is carried out in 1,4-dioxane, tetrahydrofuran (THF), water (H₂O), or mixtures thereof.

38. The method of any one of claims 27-36, wherein the reaction is carried out in 1,4-dioxane, toluene, or mixtures thereof.

39. The method of any one of claims 27-38, wherein the compound of formula (Ic) is prepared by the method of claim 1 or claim 2.

40. The method of claim 39, wherein the compound of formula (Ic) prepared by the method of claim 1 or 2 is reacted with the compound of formula (Id) without isolating the compound of formula (Ic).

41. A method of purifying a compound of formula (I): 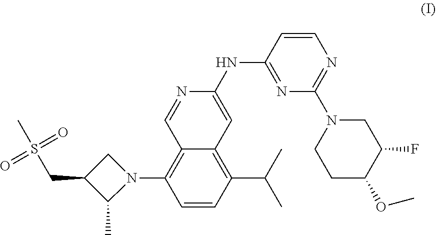 [SMILES: CO[C@@H]1CCN(c2nccc(Nc3cc4c(C(C)C)ccc(N5C[C@H](CS(C)(=O)=O)[C@H]5C)c4cn3)n2)C[C@@H]1F]
to form a crystallized product, comprising recrystallizing a compound of formula (I) in a solvent system (e.g., in a solvent system comprising dimethyl sulfoxide (DMSO) and ethanol).

42. A method of purifying a compound of formula (I): 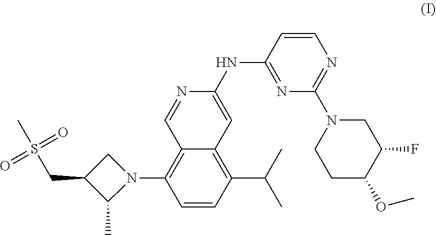 [SMILES: CO[C@@H]1CCN(c2nccc(Nc3cc4c(C(C)C)ccc(N5C[C@H](CS(C)(=O)=O)[C@H]5C)c4cn3)n2)C[C@@H]1F]
to form a crystallized product, comprising recrystallizing a compound of formula (I) in a solvent system (e.g., in a solvent system comprising dimethyl sulfoxide (DMSO) and water).

43. The method of claim 41 or claim 42, wherein the compound of formula (I) is isolated as a wet cake.

44. The method of claim 43, wherein the compound of formula (I) is obtained from the method of any one of claims 27-40.
